(19) United States
(12) Patent Application Publication
(10) Pub. No.: US 2008/0008760 A1
(43) Pub. Date: Jan. 10, 2008

(54) Functionalized carbon nanotubes, a process for preparing the same and their use in medicinal chemistry

(76) Inventors: Alberto Bianco, Strasbourg (FR); Davide Pantarotto, Porcia (IT); Maurizio Prato, Trieste (IT)

Correspondence Address: YOUNG & THOMPSON, 745 SOUTH 23RD STREET, 2ND FLOOR, ARLINGTON, VA, 22202, US

(21) Appl. No.: 11/249,328

(22) Filed: Oct. 14, 2005

Related U.S. Application Data
(63) Continuation-in-part of application No. PCT/EP04/03829, filed on Apr. 9, 2004.
Application No. PCT/EP04/03829 is a continuation-in-part of application No. PCT/EP03/03838, filed on Apr. 14, 2003.

(51) Int. Cl. D01F 9/12; A61K 38/10; A61K 38/07; A61K 38/08; C07D 403/14

(52) U.S. Cl. 424/489; 423/447.2; 977/746; 546/276.7; 530/328; 530/329; 530/330; 548/416; 977/906

(57) ABSTRACT

The present invention relates to functionalized carbon nanotubes, a process for preparing the same and their use, in particular in medicinal chemistry and more particularly in immunology.

The present invention relates to functionalized carbon nanotubes, a process for preparing the same and their use, in particular in medicinal chemistry and more particularly in immunology.

Due to their exceptional combination of mechanical, thermal, chemical, and electronic properties, single-walled (SWNT) and multi-walled carbon nanotubes (MWNT) are considered as unique materials, with very promising future applications, especially in the field of nanotechnology, nanoelectronics, composite materials and medicinal chemistry.

So far, potential biological applications of carbon nanotubes (CNT) have been very little explored.

The main difficulty to integrate such materials into biological systems derives from their lack of solubility in physiological solutions.

To extend the applications of carbon nanotubes in medicinal chemistry, water soluble samples are in demand. Very recently, it has been shown that carbon nanotubes can be solubilised in aqueous solution by a wrapping approach using starch and poly(vinylpyrrolidone) or attaching monoamine terminated poly(ethyleneoxide), glucosamine or crown ethers to the carboxylic groups of the oxidized SWNTs.

Soluble full-length carbon nanotubes have been recently achieved by side-wall organic functionalization. This type of solubilisation makes their manipulation and incorporation in different materials easier. However, the side-wall functionalization carried up to now is such that non reactive groups have been linked to the nanotubes, thus not enabling the link of molecules of biological interests.

Furthermore covalent modification has the disadvantage that it impairs the physical properties of carbon nanotubes.

Up to now, no full-length functionalized carbon nanotubes which are soluble in a wide range of organic solvents and in physiological solutions and which have interesting immunological properties have been described.

One of the aspects of the invention is to provide carbon nanotubes which are functionalized with peptides and which are biocompatible.

Another aspect of the invention is to provide a process for preparing full-length functionalized carbon nanotubes.

Another aspect of the invention is to provide substantially homogeneous solutions of functionalized carbon nanotubes.

Another aspect of the invention is to provide functionalized carbon nanotubes enabling to monitor the type of elicited immune response.

All these aims are achieved by a functionalized carbon nanotube, the surface of which carries covalently bound reactive and/or activable functional groups which are homogeneously distributed on said surface, said functionalized carbon nanotube being substantially intact and soluble in organic and/or aqueous solvents.

The expression “carbon nanotubes” refers to molecules constituted only of carbon atoms arranged in a cylinder, said cylinder being characterized by a defined length and diameter. The carbon nanotube is similar to a rolled up graphite plane, thus forming a graphite cylinder; the side-wall carbon atoms of the cylinder are arranged in order to form fused benzene rings, as in planar graphite. The cylinder is closed at its extremities; in the closed extremities, which are similar to fullerenes, five carbon rings are fused to benzene rings (Niyogi S. et al. Acc. Chem. Res. (2002) 35:1105-1113).

The expression “functionalized carbon nanotubes” refers to carbon nanotubes which have been modified by a chemical reaction which results in the addition of an organic appendage to a benzene ring of the graphite cylinder.

The expression “the surface of the carbon nanotube carries covalently bound functional groups” means that the external surface of the graphite cylinder is modified by a chemical reaction to link through a stable covalent bond an organic appendage defined as a functional group.

The expression “reactive and/or activable functional groups” means that the functional group presents itself a second site that can be subjected to a chemical reaction, such as an addition or a substitution, because it is in an active form ready to form a covalent bond with another molecule, or, if it is an unreactive functional group it can be rendered active by a chemical reaction which uncovers a site which can be subjected to a chemical reaction, such as an addition or a substitution.

It means in particular that the binding of functional groups does not come from intrinsic or induced effects.

The expression “homogeneously distributed” means that the functional groups are statistically distributed all along the surface of the carbon nanotube and not simply concentrated on a part of it, such as the extremities of the carbon nanotube. In addition, there is a ratio between the number of functional groups and the number of carbon atom of the carbon nanotube, in particular there is 1 functional group per about 50 to about 1000 carbon atoms of the carbon nanotube, more particularly there is 1 functional group per about 100 carbon atoms of the carbon nanotube.

The expression “substantially intact” means that there is a very low amount of defects on the surface, and no shortening of the carbon nanotubes, due to the oxidation of the carbon atoms of the extremities of the carbon nanotubes into carboxylic acids.

The expression “substantially soluble in organic solvents” means that the functionalized carbon nanotubes can be solubilized in organic solvents without any formation of a precipitate upon storage, due to aggregation phenomena.

The expression “substantially soluble in aqueous solvents” means that the functionalized carbon nanotubes of the invention can be solubilized in pure water or buffer solutions without any formation of a precipitate upon storage, due to aggregation phenomena.

The functionalized carbon nanotubes of the invention can be substantially soluble in pure organic solvents or in mixtures of protic organic solvents and aqueous solutions.

The functionalized carbon nanotubes of the invention can be a single-walled (SWNT) or a multi-walled carbon nanotubes (MWNT).

The single-walled carbon nanotubes (SWNT) are for instance defined in Ajayan, PM & Iijima S. Nature (1993) 361:333-334; Rao CNR. et al. Chem. Phys. Chem. (2001) 2:78-105.

The multi-walled carbon nanotubes are for instance defined in Iijima, S. Nature (1991) 354:56-58; Rao CNR. et al. Chem. Phys. Chem. (2001) 2:78-105.

According to an advantageous embodiment of the invention, the solvents in which the carbon nanotubes of the invention are soluble, are selected from a group comprising dimethylformamide, dichloromethane, chloroform, acetonitrile, dimethylsulfoxide, methanol, ethanol, toluene, isopropanol, 1,2-dichloroethane, N-methylpyrrolidone, tetrahydrofuran.

According to an advantageous embodiment, the functionalized carbon nanotubes of the invention have the following general formula:
[Cₙ]—Xₘ
wherein:
Cₙ are surface carbons of a substantially cylindrical carbon nanotube of substantially constant diameter, said diameter being from about 0.5 to about 50 nm, in particular from about 0.5 to 5 nm for SWNTs and from about 20 to about 50 nm for MWNTs,
X represents one or several functional groups, identical or different, each functional group comprising at least one effective group,
n is an integer from about 3.10³ to about 3.10⁶,
m is an integer from about 0.001 n to about 0.1 n, there are from about 2.10⁻¹¹ moles to about 2.10⁻⁹ moles of X functional groups per cm² of carbon nanotube surface.

The carbon nanotubes include those having a length to diameter ratio greater than 5 and a diameter of less than 0.2 μm, preferably less than 0.05 μm.

En substituted carbon nanotubes, the surface atoms Cₙ are reacted. Most carbon atoms in the surface layer are basal plane carbons, such as carbons constitutive of benzene rings. In the prior art, basal plane carbons are generally considered to be relatively inert to chemical attack, except those which stand at defect sites or which are analogous to the edge carbon atoms of a graphite plane.

The carbon atoms of the extremities of carbon nanotubes may include carbon atoms exposed at defects sites and edge carbon atoms.

According to an advantageous embodiment, the invention relates to an aqueous or organic solution containing functionalized carbon nanotubes wherein the distribution of so the length range of the carbon nanotubes is substantially the same as the distribution of the length range of the carbon nanotubes before functionalization.

The length of the carbon nanotubes is advantageously chosen in the range from about 20 nm to about 20 μm.

The distribution of functional groups per cm² of carbon nanotube surface which is advantageously of 2.10⁻¹¹ moles to 2.10⁻⁹ moles can be determined by DSC (differential scanning calorimetry), TGA (thermo gravimetric assay), titrations and spectrophotometric measurements.

Its homogeneity can be determined by high resolution transmission electron microscopy (TEM), provided the resolution is sufficient to see the electron density of the carbon nanotube surface and of the functional groups it carries, or by NMR (nuclear magnetic resonance) spectroscopy, provided labelled atoms, such as ¹⁵N, ¹³C or ²H, are present in functional groups.

The parameters involved in the higher and lower values of the range of the distribution of functional groups per cm² of carbon nanotube surface are the curvature of the carbon nanotube, the reaction time, the temperature of the reaction, the chemical stability of the reagents and the solvent.

The carbon nanotubes of the invention are substantially pure and do not contain amorphous or pyrolytically deposited carbon, carbon particles, or fullerenes, and are in particular devoid of metals such as Fe, Ni, Co, that are generally used as catalysts in the production of carbon nanotubes.

According to another advantageous embodiment of the invention, e represents two different functional groups, X¹ and X², said functionalized nanotube corresponding to the following formula:
[Cₙ]—[X¹ₘ₁][X²ₘ₂]

wherein, independently from each other, m₁ and m₂ represent integer from about 0.001 n to about 0.1 n.

According to a further advantageous embodiment of the invention, X represents at least one functional group comprising two effective groups, identical or different.

According to another embodiment, in an advantageous group of functionalized carbon nanotubes of the invention, X represents one or several substituted pyrrolidine rings, identical or different, and the functionalized carbon nanotubes reply to the following general formula (I): 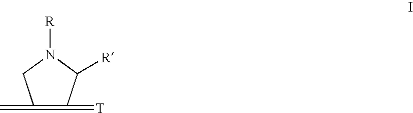
wherein T represents a carbon nanotube, and independently from each other R and R′ represent —H or a group of formula -M-Y-(Z)ₐ-(P)_{b}, wherein a represents 0 or 1 and b represents an integer from 0 to 8, preferably 0, 1, or 2, P representing identical or different groups when b is greater than 1, provided R and R′ cannot simultaneously represent H, and:
M is a spacer group from about 1 to about 100 atoms, such as a group selected from the list comprising —(CH₂)ᵣ— or —(CH₂—CH₂—O)ᵣ—CH₂—CH₂—, wherein r is an integer from 1 to 20;
Y is a reactive group when a=b=0, such as a group selected from the list comprising —OH, —NH₂, —COOH, —SH, —CHO, a ketone such as —COCH₃, an azide or a halide; or derived from a reactive group, when a or b is different from 0, such as a group selected from the list comprising —O—, —NH—, —COO—, —S—, —CH═, —CH₂—, —CCₖH₂ₖ₊₁—, wherein k is an integer from 1 to 10, in particular —CCH₃═, or —CHCₖH₂ₖ₊₁—, wherein k is an integer from 1 to 10, in particular —CHCH₃—;
Z is a linker group, liable to be linked to at least one P group, and if need be to release said P group, such as a group of one of the following formulae when a=1 and b=0: 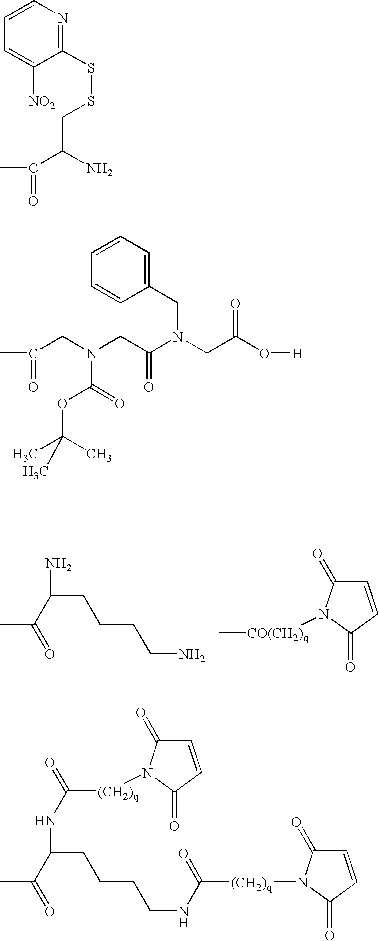
   wherein q is an integer from 1 to 10; or of the corresponding following formula when a=1 and b=1 or 2: 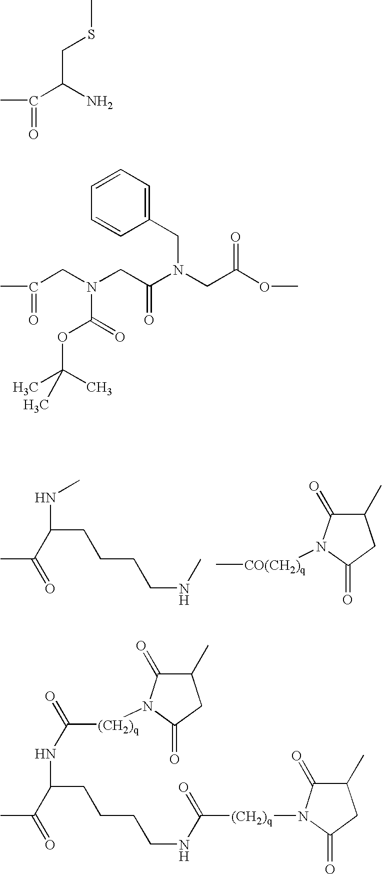
wherein q is an integer from 1 to 10;
P is an effective group allowing spectroscopic detection of said functionalized carbon nanotube, such as a fluorophore, such as FITC, a chelating agent, such as DTPA, or an active molecule, liable to induce a biological effect, such as an amino acid, a peptide, a pseudopeptide, a protein, such as an enzyme or an antibody, a nucleic acid, a carbohydrate, or a drug,
if appropriate at least one of Y, Z, or P groups, can be substituted by a capping group, such as CH₃CO— (acetyl), methyl, or ethyl, or benzylcarbonyl, or a protecting group such as methyl, ethyl, benzyl, tert-butyl, trityl, 3-nitro-2-pyridylsulfenyl, tert-butyloxycarbonyl (Boc), fluorenylmethyloxycarbonyl (Fmoc), benzyloxycarbonyl (Cbz), benzoyl (Bz), trimethylsilylethyloxycarbonyl, phtalimide, dimethylacetal, diethylacetal, or 1,3-dioxolane.

The pyrrolidine ring has the advantage of being a stable and robust cyclic molecule, presenting a nitrogen atom which can bear a spacer group at the end of which a reactive group can be present or inserted.

The expression “Y is a reactive group” means that Y represents a heteroatom, ready to undertake a chemical reaction to form a new covalent bond.

The expression “M is a spacer group” means that M is a linear organic chain which keeps separate the pyrrolidine on the carbon nanotube from the reactive function Y.

The expression “Y is derived from a reactive group” means that Y is a heteroatom or a functional group which has been modified by a chemical reaction generating a new covalent bond.

It is clear from the preceeding description, that —O— is derived from the reactive group —OH, —NH is derived from the reactive group —NH₂, —COO— is derived from the reactive group —COOH, —S— is derived from the reactive group —SH, —CH═ and —CH₂— are derived from the reactive group —CHO, —CCₖH₂ₖ₊₁— and —CHCₖH₂ₖ₊₁— are derived from the reactive group: ketone, and in particular —CCH₃═ and —CHCH₃— are derived from the reactive group —COCH₃.

As to the azide, it is a protected group.

As to the halide, the corresponding derived group can be —NH—, —O—, —S—, —COO—, or an azide.

The expression “Z is a linker group” means that Z is a chemical entity which is covalently linked to Y and allows the coupling of P, and which is resistant to the chemical reaction in the conditions of coupling for P, and which is capable of releasing P, but not of being released from Y.

According to a preferred embodiment Z refers to linker groups of the following formulae: 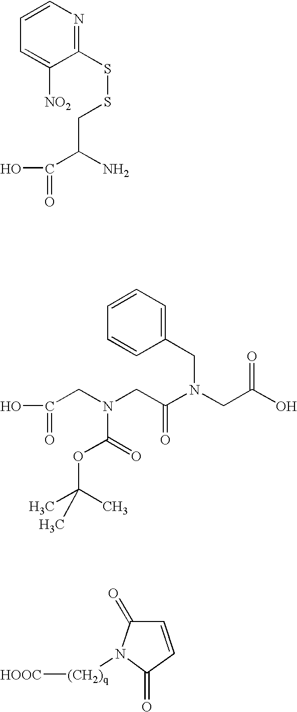
wherein q is an integer from 1 to 10;

The linker groups Z are present under varying forms depending on whether they are free, or linked to —Y— and/or linked to —P, or cleaved from —P and whether they are protected or not. The major forms of the preferred linker groups according to the invention are as follows: 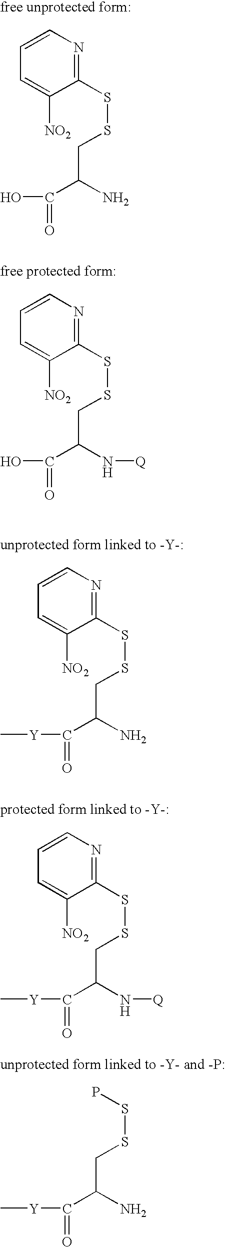 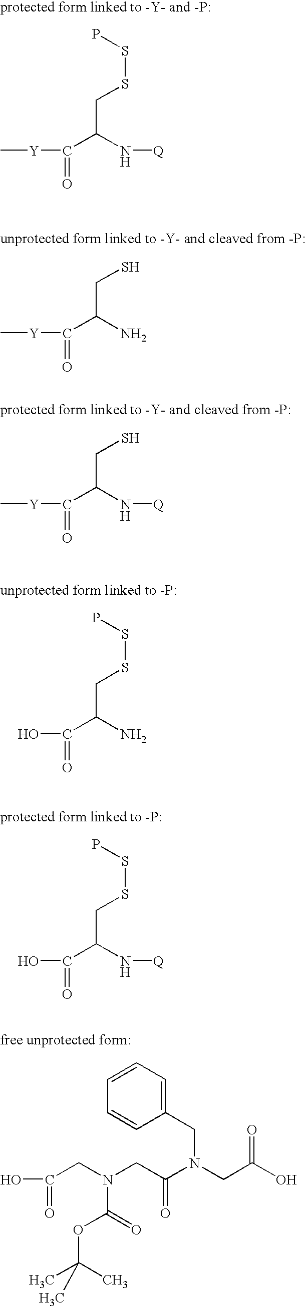 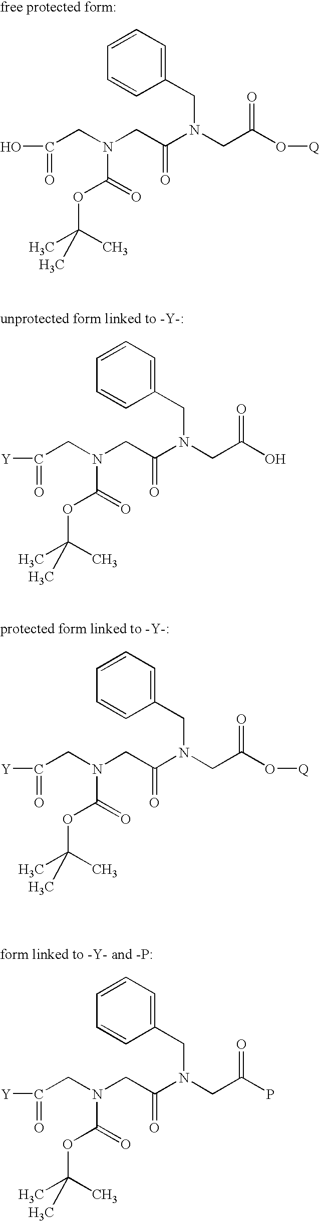 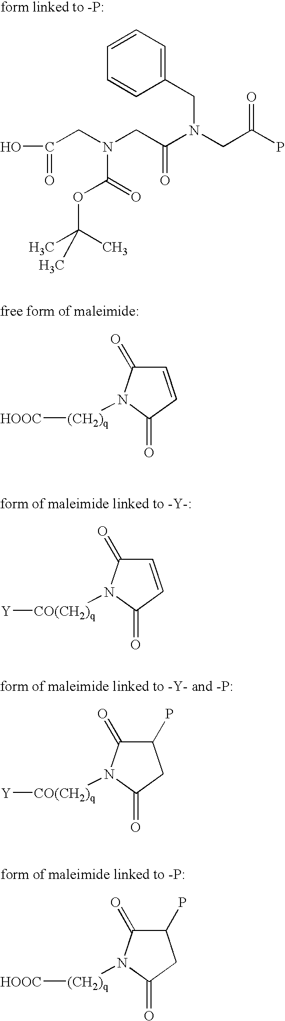
wherein q is an integer from 1 to 10, Q is a protecting group and —Y— is covalently linked to a functionalized carbon nanotube of the invention through a spacer M;

The expression “P is an effective group” means that P is a group which can confer new physical, chemical or biological properties to the carbon nanotube which carries it.

The expression “P is capable of allowing a spectroscopic detection of the carbon nanotubes” of the invention means that P is a group such as a chromophore capable of being identified by spectroscopic techniques, such as fluorescence microscopy, or nuclear magnetic resonance or FTIR (Fourier Transformed Infra-Red) spectroscopy.

The expression “active molecule liable to induce a biological effect” means that said molecule is able to modify the processes of a given biological system by establishing specific interactions with components of said biological system.

“FITC” designates fluoresceine isothiocyanate.

The expression “pseudopeptide” designates a chain of amino acids of natural or non-natural origin, which contains at least one bond, the chemical nature of which is different from an amide bond.

The expression “capping group” refers to a group capable of blocking the reactive functional group Y and which can not be removed by a chemical reaction.

The expression “protecting group” refers to a group capable of temporarily blocking the reactive functional group Y and which can be subsequently removed by a chemical reaction in order to liberate the reactive function Y for further modifications.

The nature of Z, when P is present, gives rise to two types of carbon nanotubes, those wherein P can be released or those wherein P cannot be released.

If P is present, the expression “release of P”, means that in the group -M-Y-Z-P, a cleavage might occur at the right extremity of the Z group.

When the cleavage takes place at this extremity of the Z group, then P is released.

When Z represents one of the two following molecules, and when P is present, P can be released because a cleavage can take place on the bond contiguous to the S atom, in the case of the left molecule, or P can be released from the right —COO— extremity, in the case of the right molecule.

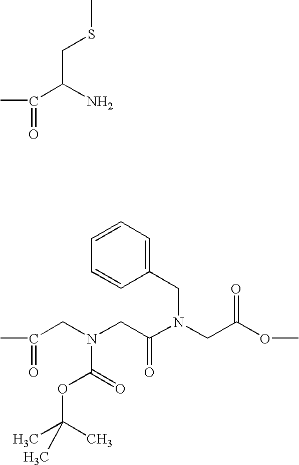

When Z represents the following molecule, and when P is present, P cannot be released, in particular under physiological conditions, such as those found in the serum, or conditions reproducing physiological conditions such as NaCl 0.15 M at pH 7.4, or PBS at pH 7.4.

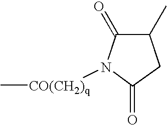

According to another embodiment of the invention, the functionalized nanotubes of the invention are such that there is generally no cleavage between M and Y—, and between Y and Z.

According to an advantageous embodiment, R represents M-Y-(Z)ₐ-(P)_{b} and R′ represents H.

According to an advantageous embodiment, M has the following formula:
—(CH₂)₂—O—(CH₂)₂—O—(CH₂)₂—

According to a preferred embodiment, X represents two different substituted pyrrolidine rings, of the following general formula (I′): 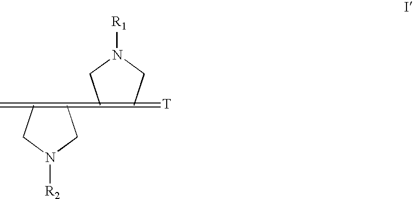
wherein T represents a carbon nanotube, R₁ and R₂ are different and represent, independently from each other, —H or a group of formula -M-Y-(Z), —(P)_{b}, wherein a represents 0 or 1 and b represents an integer from 0 to 8, preferably 0, 1, or 2, P representing identical or different groups when b is greater than 1, and:
   M is a spacer group from about 1 to about 100 atoms, such as a group selected from the list comprising —(CH₂)ᵣ— or —(CH₂—CH₂—O)ᵣ—CH₂—CH₂—, wherein r is an integer from 1 to 20;
   Y is a reactive group when a=b=0, such as a group selected from the list comprising —OH, —NH₂, —COOH, —SH, —CHO, a ketone such as —COCH₃, an azide or a halide; or derived from a reactive group, when a or b is different from 0, such as a group selected from the list comprising —O—, —NH—, —COO—, —S—, —CH═, —CH₂—, —CCₖH₂ₖ₊₁—, wherein k is an integer from 1 to 10, in particular —CCH₃═, or —CHCₖH₂ₖ₊₁—, wherein k is an integer from 1 to 10, in particular —CHCH₃—;
   Z is a linker group, liable to be linked to at least one P group, and if need be to release said P group, such as a group of one of the following formulae when a=1 and b=0:

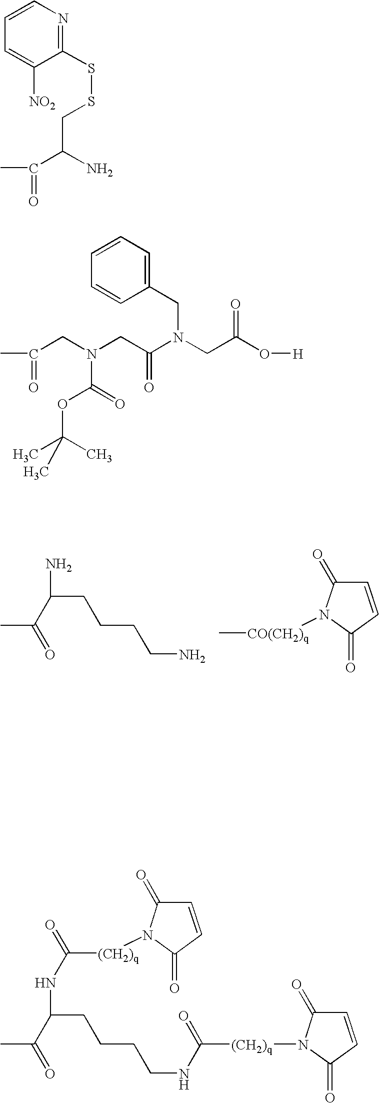

wherein q is an integer from 1 to 10;

or of the corresponding following formula when a=1 and b=1 or 2:

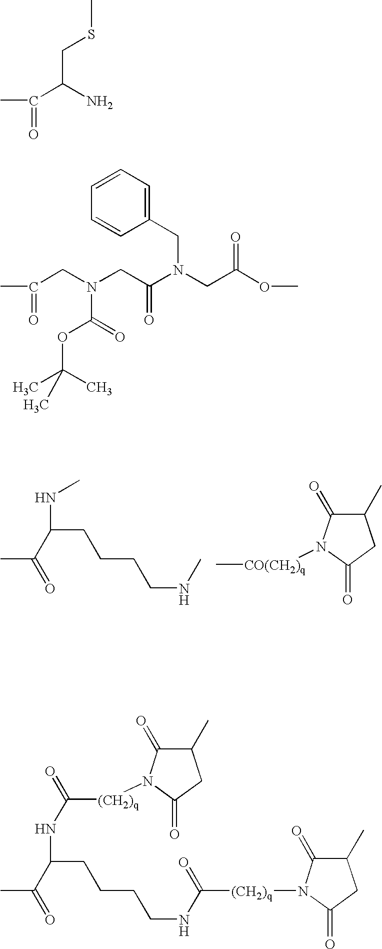

wherein q is an integer from 1 to 10;
P is an effective group allowing spectroscopic detection of said functionalized carbon nanotube, such as a fluorophore, such as FITC, a chelating agent, such as DTPA, or an active molecule, liable to induce a biological effect, such as an amino acid, a peptide, a pseudopeptide, a protein, such as an enzyme or an antibody, a nucleic acid, a carbohydrate, or a drug,

if appropriate at least one of Y, Z, or P groups, can be substituted by a capping group, such as CH₃CO— (acetyl), methyl, or ethyl, or benzylcarbonyl, or a protecting group such as methyl, ethyl, benzyl, tert-butyl, trityl, 3-nitro-2-pyridylsulfenyl, tert-butyloxycarbonyl (Boc), fluorenylmethyloxycarbonyl (Fmoc), benzyloxycarbonyl (Cbz), benzoyl (Bz), trimethylsilylethyloxycarbonyl, phtalimide, dimethylacetal, diethylacetal, or 1,3-dioxolane.

In an advantageous embodiment of the invention, the functionalized carbon nanotubes are such that a=b=0 and Y is a reactive group selected from the list comprising —OH, —NH₂, —COOH, —SH, —CHO, a ketone, such as —COCH₃, an azide, or a halide, in particular —NH₂, said functionalized carbon nanotube being, if appropriate, substituted by a capping or a protecting group, such as defined above, in particular a Bz, Boc or acetyl group, and being for instance a functionalized carbon nanotube of one of the following formulae:

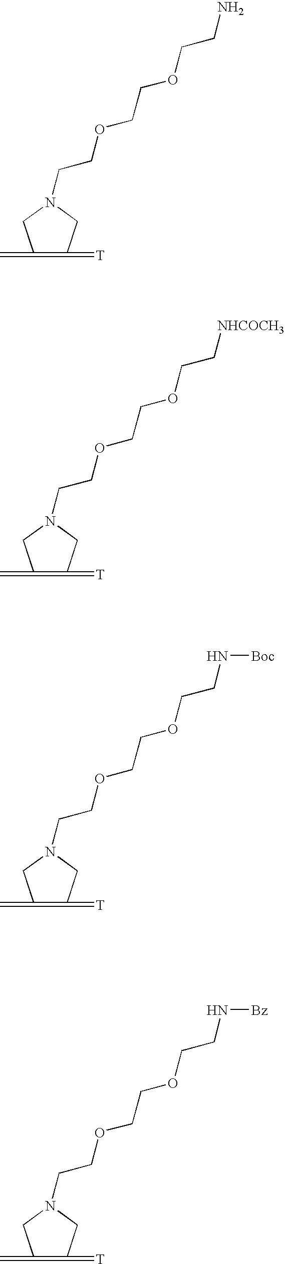

The functionalized carbon nanotubes of the invention wherein a=b=0, correspond to an advantageous group of the invention, in which it is possible to bind covalently an effective group, and advantageously an amino acid or a peptide.

These compounds are highly soluble in organic solvents and aqueous solutions. In particular, the compound on the left is ready for the coupling of a linker Z and/or of a P group. The compound in the middle with the amino function blocked by a capping group can be used as a control in biological assays, since it is not endowed with any biological activity. The compound on the right, which carries a Boc protecting group, is the precursor of the left molecule, after cleavage of the Boc protecting group.

In another advantageous embodiment of the invention, the functionalized carbon nanotubes are such that a=1 and b=0, Y is derived from a reactive group and selected from the list comprising —O—, —NH—, —COO—, —S—, —CH═, —CH₂—, —CCₖH₂ₖ₊₁—, wherein k is an integer from 1 to 10, in particular —CCH₃═, or —CHCₖH₂ₖ₊₁—, wherein k is an integer from 1 to 10, in particular —CHCH₃—, and Z is as defined above and represents in particular the group of the following formula: 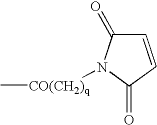
or of the following formula: 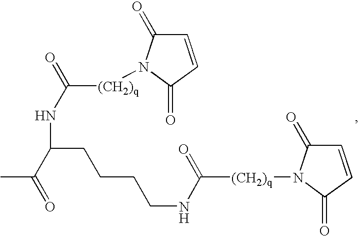
wherein q is an integer from 1 to 10, said functionalized carbon nanotube being if appropriate substituted by a protecting group, such as defined in claim 5, and being for instance the functionalized carbon nanotube of the following formula: 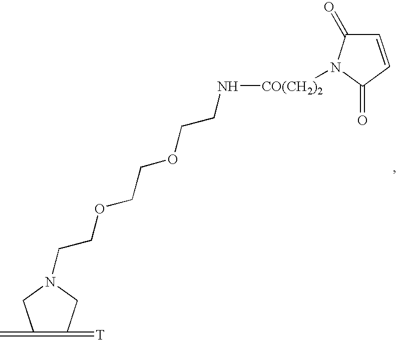
or of the following formula:

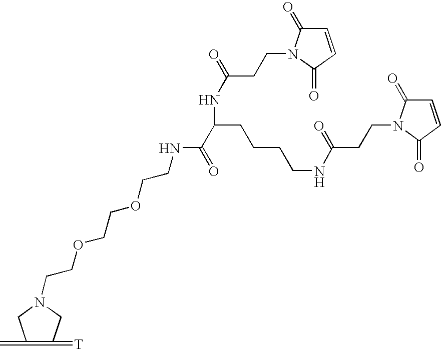

The functionalized nanotubes of the invention wherein a=1 and b=0, correspond to an advantageous group of the invention, on which it is possible to bind covalently an effective group and advantageously an amino acid or a peptide.

This compound can be linked to a P group through a selective chemical ligation. In particular the maleimido group permits the direct formation of a covalent bond by the addition of a molecule which comprises a free thiol group.

In another advantageous embodiment of the invention, the functionalized carbon nanotubes are such that a=0 and b=1, Y is derived from a reactive group and selected from the list comprising —O—, —NH—, —COO—, —S—, —CH═, —CH₂—, —CCₖH₂ₖ₊₁—, wherein k is an integer from 1 to 10, in particular —CCH₃═, or —CHCₖH₂ₖ₊₁—, wherein k is an integer from 1 to 10, in particular —CHCH₃—, and P is an effective group or an active molecule, such as defined above, in particular FITC, an amino acid, such as glycine, or a peptide, such as the peptide H-Lys-Gly-Tyr-Tyr-Gly-OH, or DTPA, said functionalized carbon nanotube being if appropriate substituted by a protecting group as defined above, such as Fmoc, and being for instance a functionalized carbon nanotube of one of the following formulae:

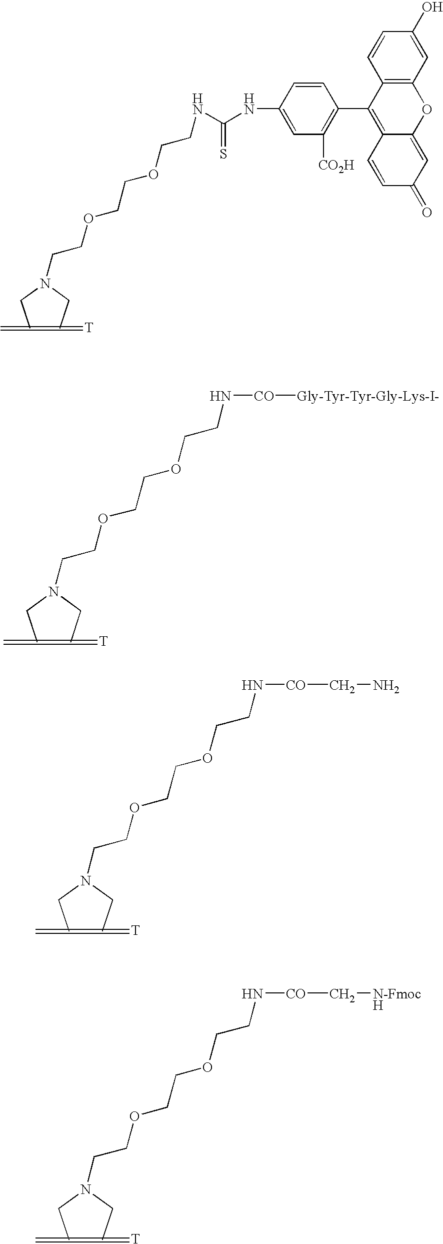

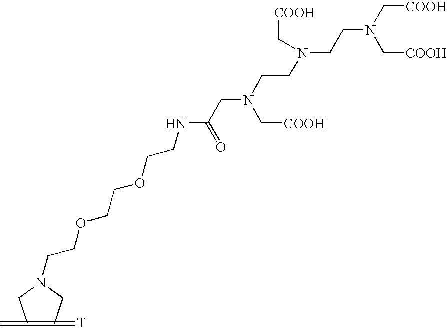

The carbon nanotube functionalized with FITC presents a useful probe for its detection by fluorescence microscopy. The pentapeptide H-Lys-Gly-Tyr-Tyr-Gly-OH contains a subpart of a protein belonging to the TNF (Tumor Necrosis Factor) family, proteins of this family being involved in autoimmune response, and being liable to be used to modulate cellular interaction. The carbon nanotube functionalized with this pentapeptide can therefore be used for modulating cellular interactions. The carbon nanotube with the glycine can be used as a starting material for a step-by-step peptide synthesis. The Fmoc protected form is a precursor form of the previous functionalized carbon nanotube.

In another advantageous embodiment of the invention, the functionalized carbon nanotubes are such that a=1 and b=1, Y is derived from a reactive group and selected from the list comprising —O—, —NH—, —COO—, —S—, —CH═, —CH₂—, —CCₖH₂ₖ₊₁═, wherein k is an integer from 1 to 10, in particular —CCH₃═, or —CHCₖH₂ₖ₊₁—, wherein k is an integer from 1 to 10, in particular —CHCH₃—, Z is as defined above and represents in particular the group of the following formula: 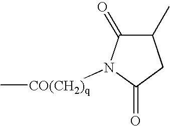
wherein q is an integer from 1 to 10, and P is as defined above, in particular a peptide, such as the peptide Acetyl-Cys-Gly-Ser-Gly-Val-Arg-Gly-Asp-Phe-Gly-Ser-Leu-Ala-Pro-Arg-Val-Ala-Arg-Gln-Leu-OH, said functionalized carbon nanotubes being if appropriate substituted by a protecting group, such as defined above, and being for instance the functionalized carbon nanotubes of the following formula:

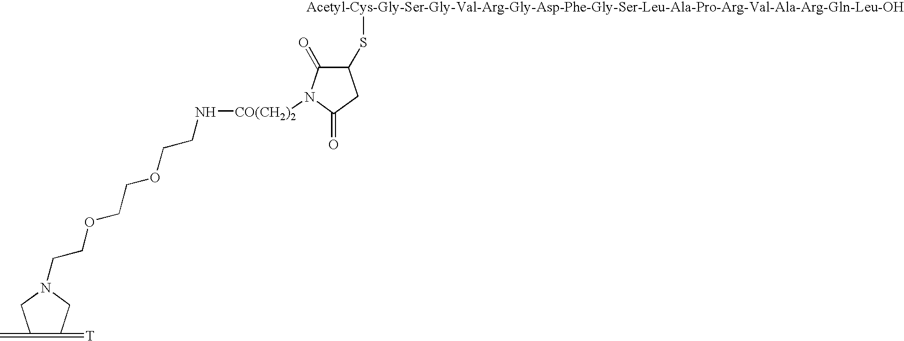

This carbon nanotube presents a B-cell epitope corresponding to the sequence 141-159 of the VP1 coat protein from the foot and mouth disease virus (FMDV), it is capable of inducing the production of neutralizing antibodies upon immunization of animals such as mice for instance.

The present invention also relates to a functionalized carbon nanotube as defined above, wherein a=1 and b=2, that is wherein independently of each other R and R′ represent —H or a group of formula M-Y-Z-(P)₂ or M-Y-Z-(PP′), provided R and R′ cannot simultaneously represent —H, Y is derived from a reactive group and selected from the list comprising —O—, —NH—, —COO—, —S—, —CH═, —CH₂—, —CCₖH₂ₖ₊₁═, wherein k is an integer from 1 to 10, in particular —CCH₃═, or —CHCₖH₂ₖ₊₁—, wherein k is an integer from 1 to 10, in particular —CHCH₃—, Z is as defined above and represents in particular the group of the following formula:

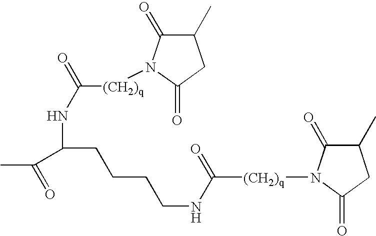

wherein q is an integer from 1 to 10, P and P′ are different and represent an effective group allowing spectroscopic detection of said functionalized carbon nanotube, such as a fluorophore, such as FITC, a chelating agent, such as DTPA, or an active molecule, liable to induce a biological effect, such as an amino acid, a peptide, a pseudopeptide, a protein, such as an enzyme or an antibody, a nucleic acid, a carbohydrate, or a drug, in particular P and P′ represent a peptide, such as the peptide Acetyl-Cys-Gly-Ser-Gly-Val-Arg-Gly-Asp-Phe-Gly-Ser-Leu-Ala-Pro-Arg-Val-Ala-Arg-Gln-Leu-OH, said functionalized carbon nanotube being if appropriate substituted by a protecting group, such as defined above, and being for instance the functionalized carbon nanotube of the following formula:

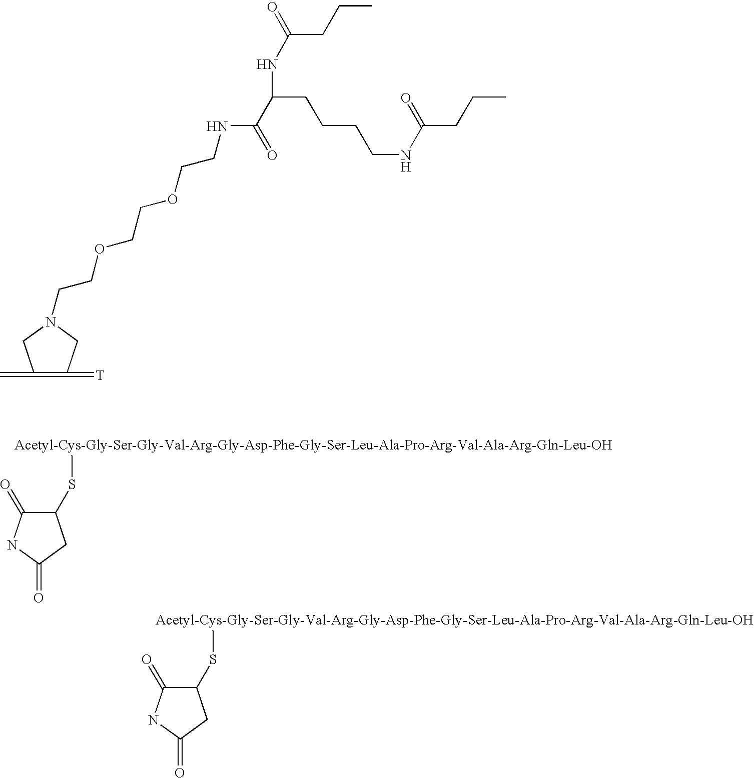

In another advantageous embodiment of the invention, the functionalized carbon nanotubes are such that b=1, P is a peptide or a protein, said peptide or protein comprising in particular a B cell epitope or a T cell epitope, such as a T helper epitope or a T cytotoxic epitope, or a mixture thereof.

The B or T cell nature of a given epitope can be assessed as follows:
in the case of a carbon nanotube functionalized with a putative T cell epitope, the functionalized nanotube can be administered, optionally in association with an adjuvant, to an animal, in particular a mouse; T cells, in particular CD4+ (helper) or CD8+ (cytotoxic) T cells, are then purified from said animal according to methods well known to the man skilled in the art, and used to verify if said functionalized nanotube is capable of activating said T cells; the activation of T cells can be assayed by several methods well known to the man skilled in the art, such as proliferation assays, cytokine production assays or membrane marker expression assays;
in the case of carbon nanotube functionalized with a putative B cell epitope, the functionalized nanotube is administered at least once to an animal, in particular a mouse; antibodies directed against the putative B cell epitope are then searched for in blood, plasma or serum of said animal, with methods well known to the man skilled in the art, such as an ELISA test for example.

The invention also relates to a process for preparing a functionalized carbon nanotube of the following formula I: 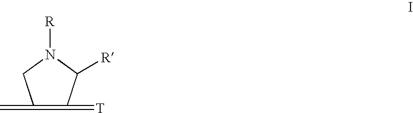
wherein T represents a carbon nanotube and independently from each other R and R′ represent —H or a group of formula -M-Y, provided R and R′ cannot simultaneously represent H, wherein:
-M- is a spacer group from about 1 to about 100 atoms, such as a group selected from the list comprising —(CH₂)ᵣ— or —(CH₂—CH₂—O)ᵣ—CH₂—CH₂—, wherein r is an integer from 1 to 20;
—Y is a reactive group, such as a group selected from the list comprising, —OH, —NH₂, —COOH, —SH, —CHO, a ketone such as —COCH₃, an azide, a halide, if appropriate protected, such as —O-Q, —NH-Q, —COO-Q, —S-Q, —CH(OQ)₂, 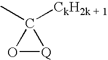
   wherein k is an integer from 1 to 10, in particular 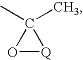
   wherein Q is a protecting group or forms a protecting group with the adjacent atoms to which it is linked;
   said process comprising the following step:
adding, to a carbon nanotube, the compounds R′-CHO and R—NH—CHR″—COOR′″ by a 1,3-dipolar cycloaddition, wherein:
   R and R′ are as defined above;
   R″ is —H or an amino acid side-chain;
   R′″ is —H, an allyl group of 1 to 5 carbon atoms, a (CH₂CH₂O)ₜ—CH₃
   group, wherein t is an integer from 1 to 20, or an aromatic group; to obtain a functionalized carbon nanotube of formula I, if appropriate protected; if necessary, deprotecting the functionalized carbon nanotube of formula I, to obtain an unprotected functionalized carbon nanotube of formula I.

Optionally, carbon nanotubes can be fluorinated in a first step, and then in a second step, the fluorine atom can be substituted with alkyl groups by treatment with alkyl lithium compounds or Grignard compounds, or the fluorine atom can be substituted by hydrazine or diamines (Khabashesku V. N. et al., Acc. Chem. Res. (2002) 35:1087-1095).

Carbon nanotubes can be also functionalized by reactive species such as nitrenes, carbenes, and radicals, through nucleophilic additions. However, the functional groups for further modification must be carefully chosen, due to the drastic conditions of some reactions, which might result in a shortening of the carbon nanotube (Hirsh A. Angew. Chem. Int. Ed. (2002) 41:1853-1859).

It appears from the preceding description that —O-Q is the protected form of —OH, —NH-Q and the azide are the protected forms of —NH₂, —COO-Q is the protected form of —COOH, —S-Q is the protected form of —SH, —CH(OQ)₂ is the protected form of —CHO, 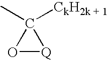
is the protected form of a ketone.

When the protected form is —CH(OQ)₂ or 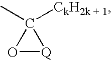
Q forms a protecting group with the adjacent atoms to which it is linked, which means that the carbonyl function of the ketone is protected as a cyclic derivative (1,3-dioxolane for instance) and that the carbonyl function of the aldehyde is protected as an acetal.

When Y is a protected reactive group, the deprotection step removes the protecting group Y, to yield the unprotected functionalized carbon nanotube of formula I.

The present invention also relates to a process for preparing a functionalized carbon nanotube of the following formula I′:

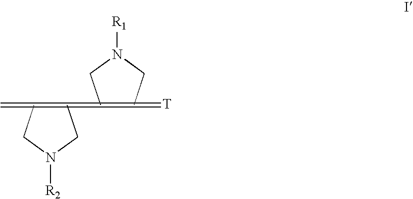

wherein T represents a carbon nanotube, R₁ and R₂ are different and represent independently from each other —H or a group of formula -M-Y, wherein:
-M- is a spacer group from about 1 to about 100 atoms, such as a group selected from the list comprising —(CH₂)ᵣ— or —(CH₂—CH₂—O)ᵣ—CH₂—CH₂—, wherein r is an integer from 1 to 20;
—Y is a reactive group, such as a group selected from the list comprising, —OH, —NH₂, —COOH, —SH, —CHO, a ketone such as —COCH₃, an azide, a halide, if appropriate protected, such as —O-Q, —NH-Q, —COO-Q, —S-Q, —CH(OQ)₂, 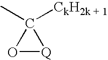
   wherein k is an integer from 1 to 10, in particular 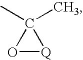
   wherein Q is a protecting group or forms a protecting group with the adjacent atoms to which it is linked;

said process comprising the following step:
adding to a carbon nanotube by a 1,3-dipolar cycloaddition, a mixture of the compounds (CH₂O)ₙ (paraformaldehyde), R₁—NH—CH₂—COOH and R₂—NH—CH₂—COOH, wherein R₁ and R₂ are different and represent independently from each other a group of formula -M-Y, to obtain a functionalized carbon nanotube of formula I′, if appropriate protected, R₁ and R₂ carrying similar or different protecting groups,
if necessary, deprotecting R₁ and/or R₂.

The invention also relates to a process for preparing a functionalized carbon nanotube of the following formula I: 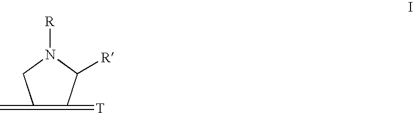
wherein T represents a carbon nanotube and independently from each other R and R′ represent —H or a group of formula -M-Y-Z, provided R and R′ cannot simultaneously represent —H, wherein:
-M- is a spacer group from about 1 to about 100 atoms, such as a group selected from the list comprising —(CH₂)ᵣ— or —(CH₂—CH₂—O)ᵣ—CH₂—CH₂—, wherein r is an integer from 1 to 20;
—Y— is a group derived from a reactive group, such as a group selected from the list comprising, —O—, —NH—, —COO—, —S—, —CH═, —CH₂—, —CCₖH_{2k+1═, wherein k is an integer from} 1 to 10, in particular —CCH₃═, or —CHCₖH₂ₖ₊₁—, wherein k is an integer from 1 to 10, in particular —CHCH₃—;
-Z is a linker group, liable to be linked to at least one P group, and if need be to release said P group, if appropriate protected by one or several capping or protecting groups -Q, identical or different, such as a group of one of the following formulae:

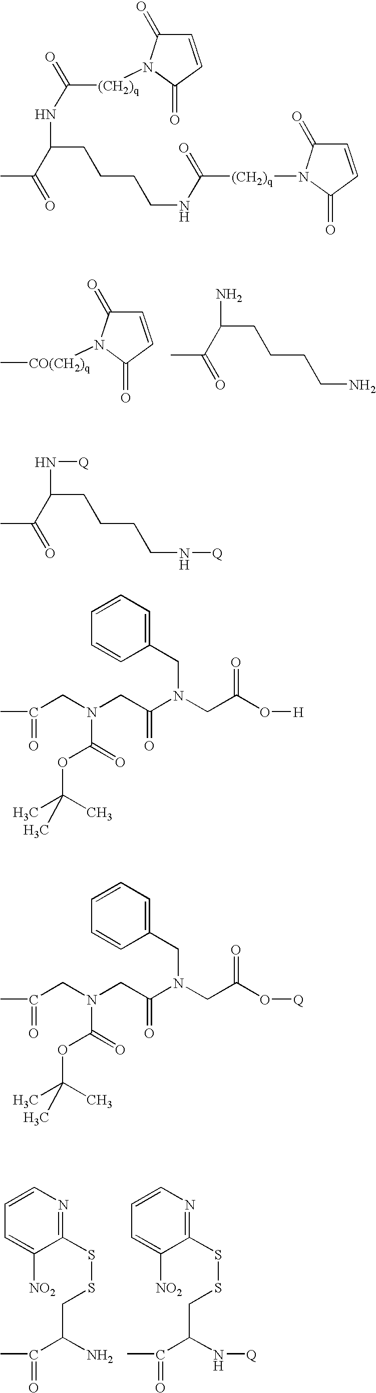

wherein q is an integer from 1 to 10; said process comprising the following steps:
adding a linker group of formula Z to a unprotected functionalized carbon nanotube of formula I wherein R and R′ represent independently from each other —H or -M-Y, -M- and —Y having the definitions above mentioned and provided that both R and R′ do not simultaneously represent H, said group Z being if appropriate protected by one or several capping or protecting groups -Q, identical or different, said group Z being for instance a linker group of one of the following formulae: 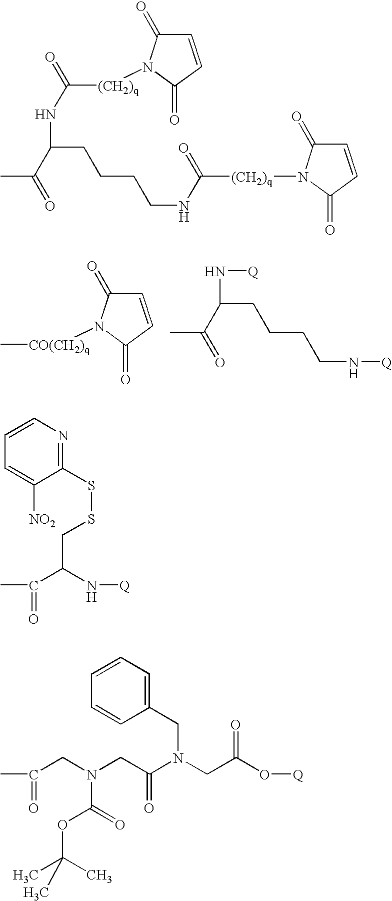
   wherein q is an integer from 1 to 10; to obtain a functionalized carbon nanotube of formula I wherein R and R′ represent independently from each other —H or -M-Y-Z and R and R′ being not simultaneously —H, if appropriate protected;

if necessary, deprotecting the functionalized carbon nanotubes of formula I, to obtain an unprotected functionalized carbon nanotubes of formula I.

The invention also relates to a process for preparing a functionalized nanotube of the following formula I: 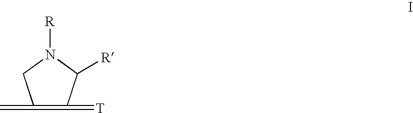
wherein T represents a carbon nanotube and independently from each other R and R′ represent —H or a group of formula -M-Y-Z-P or of formula -M-Y—P, provided R and R′ cannot simultaneously represent —H, wherein:
-M- is a spacer group from about 1 to about 100 atoms, such as a group selected from the list comprising —(CH₂)ᵣ— or —(CH₂—CH₂—O)ᵣ—CH₂—CH₂—, wherein r is an integer from 1 to 20;
—Y— is a group derived from a reactive group, such as a group selected from the list comprising, —O—, —NH—, —COO—, —S—, —CH═, —CH₂—, —CCₖH₂ₖ₊₁═, wherein k is an integer from 1 to 10, in particular —CCH₃═, or —CHCₖH₂ₖ₊₁—, wherein k is an integer from 1 to 10, in particular —CHCH₃—;
-Z- is a linker group, liable to be linked to a P group, and if need be to release said P group, such as a linker group of one of the following formulae: 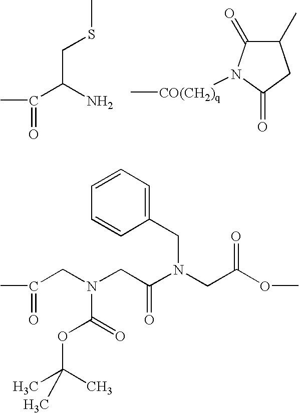
   wherein q is an integer from 1 to 10;
—P is an effective group allowing spectroscopic detection of said functionalized carbon nanotube, such as a fluorophore, such as FITC, a chelating agent, such as DTPA, or an active molecule, liable to induce a biological effect, if appropriate protected, such as an amino acid, a peptide, a pseudopeptide, a protein, such as an enzyme or an antibody, a nucleic acid, a carbohydrate, or a drug;
   said process comprising the following steps:
adding an effective group or an active molecule of formula P to an unprotected functionalized carbon nanotube of formula I wherein R and R′ represent independently from each other —H, -M-Y or -M-Y-Z, provided that R and R′ cannot simultaneously represent —H, said effective group or active molecule of formula P being if appropriate protected, such as a fluorophore, such as FITC, a chelating agent, such as DTPA, an amino acid, a peptide, a pseudopeptide, a protein, such as an enzyme or an antibody, a nucleic acid, a carbohydrate, or a drug,
or adding a group of formula Z-P, if appropriate protected, to an unprotected functionalized carbon nanotube of formula I wherein R and R′ represent independently from each other H or M-Y, provided that R and R′ cannot simultaneously represent H, to obtain a functionalized carbon nanotube of formula I, if appropriate protected;
if necessary, deprotecting the functionalized carbon nanotubes of formula I, to obtain an unprotected functionalized carbon nanotube of formula I, wherein R and R′ represent —H or a group -M-Y-Z-P or -M-Y—P, provided that R and R′ cannot simultaneously represent —H.

According to another embodiment, the functionalized nanotubes of the invention of formula I, wherein R and I or R′ represent -M-Y-Z-P can be prepared by adding Z-P to a functionalized nanotube of formula I, wherein R and/or R′ represent -M-Y.

A Z group can be added to a P group for covalently linking Z and P, the Z-P group is then linked through its Z moiety to the free Y group present on a functionalized nanotube under reaction conditions which do not cleave the Z-P bond.

The invention also relates to a process for preparing a peptide or protein functionalized carbon nanotube, of the following formula I: 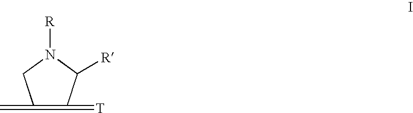
wherein T represents a carbon nanotube and independently from each other R and R′ represent H or a group of formula -M-Y—P or of formula -M-Y-Z, provided R and R′ cannot simultaneously represent —H, wherein:
-M- is a spacer group from about 1 to about 100 atoms, such as a group selected from the list comprising —(CH₂)ᵣ— or —(CH₂—CH₂—O)ᵣ—CH₂—CH₂—, wherein r is an integer from 1 to 20;
—Y— is a group derived from a reactive group, such as a group selected from the list comprising, —O—, —NH—, —COO—, —S—, —CH═, —CH₂—, —CCₖH₂ₖ₊₁═, wherein k is an integer from 1 to 10, in particular —CCH₃═, or —CHCₖH₂ₖ₊₁—, wherein k is an integer from 1 to 10, in particular —CHCH₃—;
-Z- is a linker group, in particular a group of the following formula: 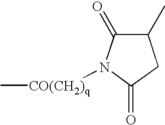
wherein q is an integer from 1 to 10;
—P is a peptide, in particular of following formula: —[OC—CHAᵢ-NH]₁—H, wherein -Aᵢ is an amino acid side-chain, i is an integer from 1 to t and t is an integer from 1 to 150, advantageously from 1 to 50;
   said process comprising the following steps:
adding a protected amino acid of the following formula:
   Q-NH—CHAᵢ-COOH
   wherein -Aᵢ is as defined above and -Q is a protecting group to a functionalized carbon nanotube of formula I, wherein R and R′ represent independently from each other —H or a group of formula -M-Y, provided that R and R′ cannot simultaneously represent —H, to obtain a functionalized carbon nanotube of the following formula II: 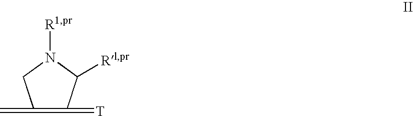
   wherein independently from each other R^{l,pr} and R′^{l,pr} represent —H or a group of formula -M-Y—OC—CHAᵢ-NH-Q, or of formula -M-Y-Z-OC—CHAᵢ-NH-Q, wherein -M-, —Y—, -Z-, -Aᵢ and -Q are as defined above;
deprotecting the functionalized carbon nanotube of formula II to obtain a functionalized carbon nanotube of the following formula II: 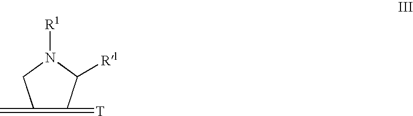
   wherein independently from each other R^{l} and R′^{l} represent —H or a group of formula -M-Y—OC—CHAᵢ-NH₂, or of formula -M-Y-Z-OC—CHAᵢ-NH₂, wherein -M-, —Y—, -Z-, and -Aᵢ are as defined above;
adding to the functionalized carbon nanotube obtained at the preceding step a protected amino acid of the following formula:
   Q-NH—CHAᵢ-COOH
   wherein -Aᵢ is as defined above and -Q is a protecting group to obtain a functionalized carbon nanotube of the following formula IV: 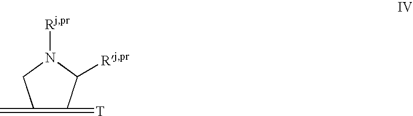
   wherein independently from each other R^{j,pr} and R′^{j,pr} represent —H or a group of formula -M-Y—[OC—CHAᵢ-NH]ⱼ-Q, or of formula -M-Y-Z-[OC—CHAᵢ-NH]ⱼ-Q, wherein -M-, —Y—, -Z-, -Aᵢ and -Q are as defined above, and j is an integer from 2 to t;
deprotecting the functionalized carbon nanotube of formula IV to obtain a functionalized carbon nanotube of the following formula V: 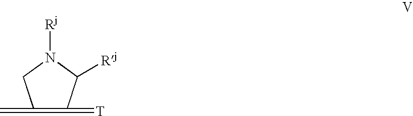
   wherein independently from each other Rⱼ and R′^{j} represent —H or a group of formula -M-Y—[OC—CHAᵢ-NH]ⱼ—H, or of formula M-Y-Z-[OC—CHAᵢ-NH]ⱼ—H, wherein -M-, —Y—, -Z-, and -Aᵢ are as defined above, and j is an integer from 2 to t;
repeating the last two steps t-1 times to obtain a peptide or protein functionalized carbon nanotube of formula I.

In this process, the peptide is synthesized step-by-step. This process is advantageously used when there is no linker group Z, since the functional group, for example NH₂, can be easily derivatized by coupling the first amino acid, protected at the N-terminus and all the other residues upon cleavage of the N-terminal protecting group. The linker in this case is not necessary due to the fact that the first peptide should remain covalently attached to the carbon nanotube.

According to another embodiment of the invention it is also possible to perform a step-by-step synthesis in the case of the presence of a maleimide junction, as a non cleavable linker, upon reaction of a N-terminal protected, C-terminal blocked, and SH-free cysteine, or of a N-protected amino thiol free derivative. After deprotection of the amine junction, the step-by-step synthesis can be processed as described above.

In the process of the invention, -Q is a capping group, such as CH₃CO— (acetyl), methyl, ethyl, or benzylcarbonyl, or a protecting group, such as a group selected from the list comprising methyl, ethyl, benzyl, tert-butyl, trityl, 3-nitro-2-pyridylsulfenyl, tert-butyloxycarbonyl (Boc), fluorenylmethyloxycarbonyl (Fmoc), benzyloxycarbonyl (Cbz), benzoyl (Bz), trimethylsilylethyloxycarbonyl, phtalimide, or ethyleneoxy.

The invention relates more particularly to a process for preparing a functionalized carbon nanotube of one of the following formulae VI, VII and VII′: 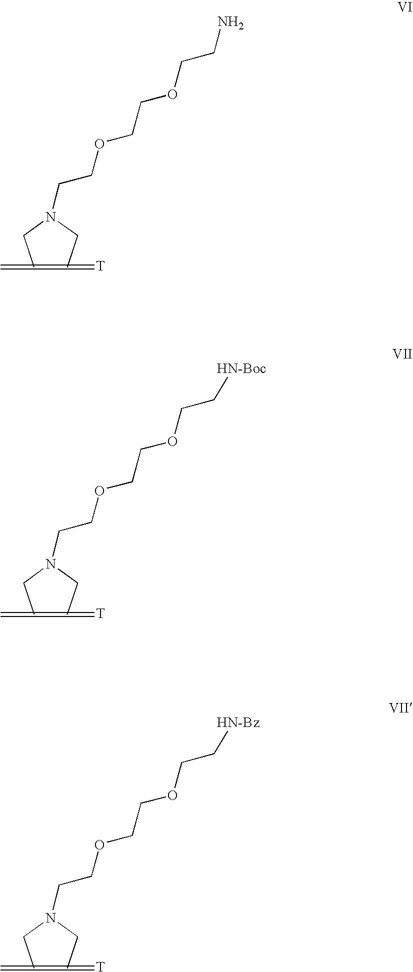
wherein T represents a carbon nanotube, Boc represents tert-butyloxycarbonyl and Bz represents benzoyl, said process comprising the following steps:
adding, to a carbon nanotube, the compounds (CH₂O)ₙ (paraformaldehyde) and Boc-NH—(CH₂—CH₂—O)₂—CH₂—CH₂—NH—CH₂—COOH or Bz-NH—(CH₂—CH₂—O)₂—CH₂—CH₂—NH—CH₂—COOH by a 1,3-dipolar cycloaddition, to obtain a protected functionalized carbon nanotube of respective formula VII or VII′;
if necessary, deprotecting the protected functionalized carbon nanotube of formula VII or VII′, to obtain an unprotected functionalized carbon nanotube of formula VI.

The invention relates more particularly to a process for preparing a functionalized carbon nanotube of the following formula VIII: 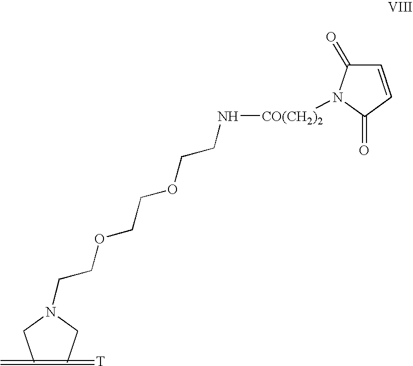
wherein T represents a carbon nanotube, said process comprising the following step:
adding, to a carbon nanotube of formula VI above defined, a compound of the following formula: 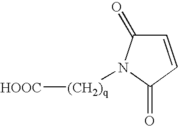
   to obtain a functionalized carbon nanotube of formula VIII.

The invention relates more particularly to a process for preparing a functionalized carbon nanotube of one of the following formulae IXa, IXb, IXc, IXd, IXe, IXf, IXg, Xb, Xc and Xf: 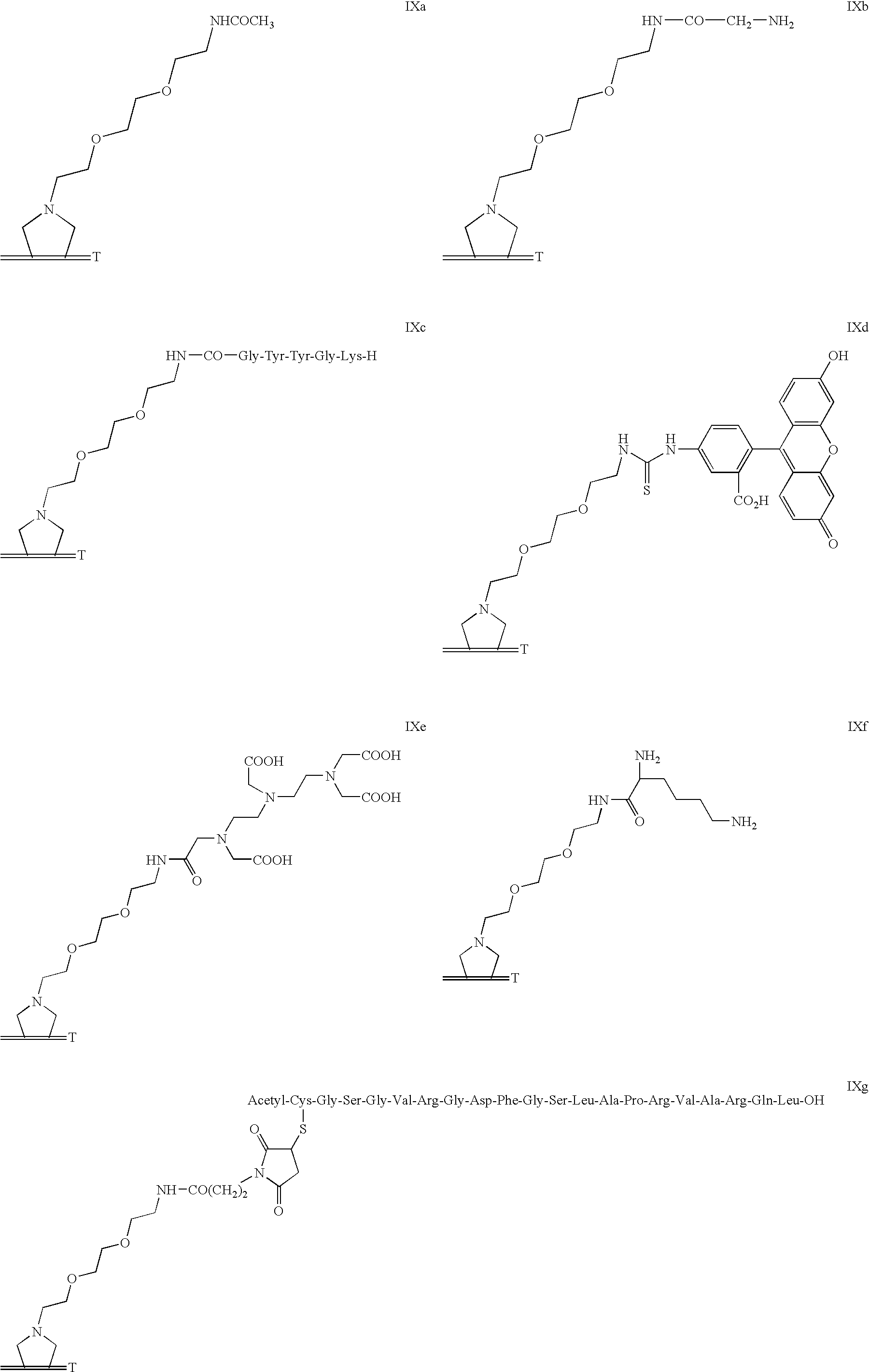 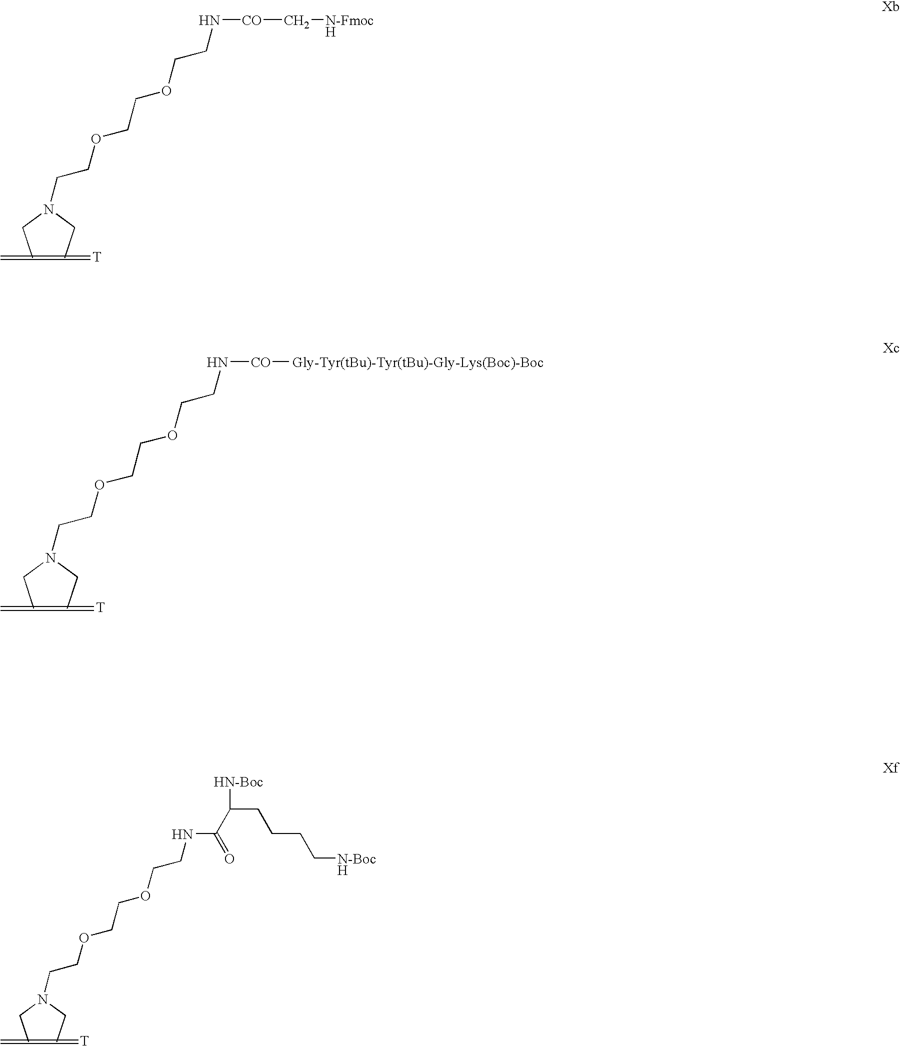
wherein T represents a carbon nanotube, Fmoc represents fluorenylmethyloxycarbonyl, tBu represents tert-butyl and Boc represents tert-butyloxycarbonyl, said process comprising the following steps:
adding,
   either to functionalized carbon nanotube of formula VI according to claim 21, a group chosen among: CH₃—COOH, Fmoc-Gly-OH, Boc-Lys(Boc)-Gly-Tyr(tBu)-Tyr(tBu)-Gly-OH, FITC, DTPA or Boc-Lys Lys(Boc)-OH,
   or to a functionalized carbon nanotube of formula VIII according to claim 22, the following group, Acetyl-Cys-Gly-Ser-Gly-Val-Arg-Gly-Asp-Phe-Gly-Ser-Leu-Ala-Pro-Arg-Val-Ala-Arg-Gln-Leu-OH,
to obtain a functionalized carbon nanotube of respective formula IXa, Xb, Xc, IXd, IXe, IXf or IXg;
if necessary, deprotecting the functionalized carbon nanotube of formula Xb, Xc, or Xf, to obtain respectively the functionalized carbon nanotube of formula IXb, IXc, or IXf.

The present invention also relates to a process for preparing a functionalized carbon nanotube of the following formulae XIa and XIb: 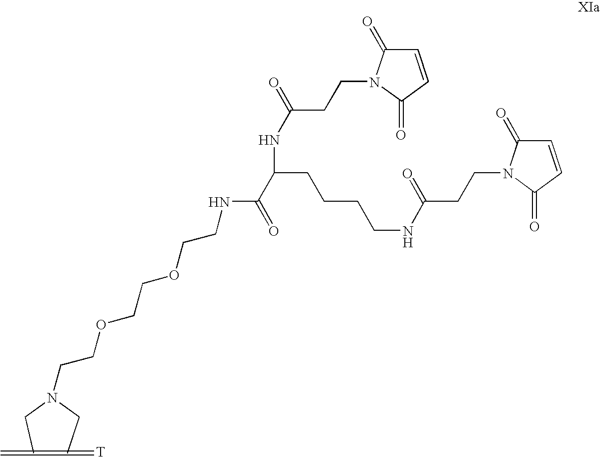 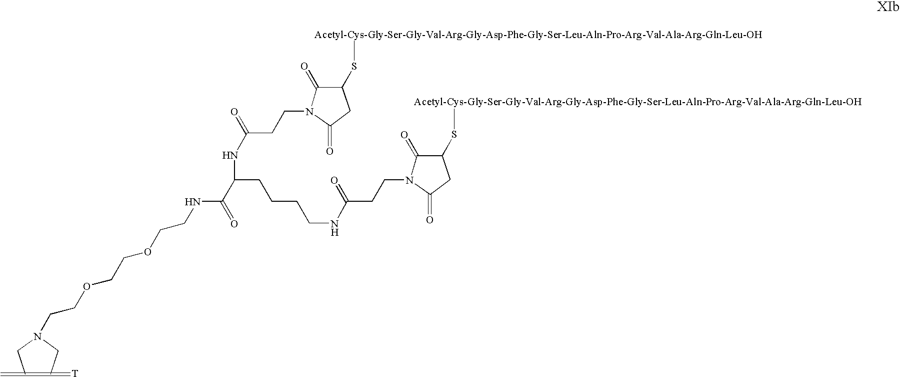
wherein T represents a carbon nanotube, said process comprising the following step:
adding, to a carbon nanotube of formula IXf, a compound of the following formula: 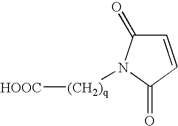
to obtain a functionalized carbon nanotube of formula XIa,
optionally adding to the functionalized nanotube of formula XIa the following group, Acetyl-Cys-Gly-Ser-Gly-Val-Arg-Gly-Asp-Phe-Gly-Ser-Leu-Ala-Pro-Arg-Val-Ala-Arg-Gln-Leu-OH, to obtain a functionalized carbon nanotube of formula XIb.

The invention also encompasses functionalized carbon nanotubes such as obtained by any of the embodiments of the process above described.

The invention also relates to a pharmaceutical composition comprising as active substance at least one functionalized carbon nanotube of the invention, said functionalized carbon nanotube being non-toxic, in association with a pharmaceutically acceptable vehicle, such as a liposome, a cyclodextrin, a microparticle, a nanoparticle, or a cell penetrating peptide.

By “non-toxic” it is meant that functionalized carbon nanotubes according to the invention present essentially no toxicity when introduced into cells. The absence of toxicity can be measured as described in Example 16 for instance. Advantageously the lack of toxicity of functionalized carbon nanotubes according to the invention is linked to their high solubility in aqueous solvents.

The functionalized nanotube according to the invention can contain an active molecule, said active molecule being liable to exert its biological effect even when covalently bound to the carbon nanotube. Advantageously the presence of several active molecules covalently bound to a single carbon nanotube can enhance the biological effect of said active molecule.

Indeed, polyvalent interactions are characterized by simultaneous binding of multiple ligands localized on a biological surface with the corresponding receptors localized on another surface. The gain on affinity by multivalent binding may have important implications on the design of new medicaments. Many copies of the same active molecule presented at the same time on the same multimeric system display high avidities as compared to the biological activities of a single monomeric unit such as discussed in Mammen et al. Angew. Chem. Int. Ed. (1998) 37:2754-2794.

The functionalized carbon nanotubes of the invention can also be used as a pharmaceutical vehicle.

As a pharmaceutical vehicle it can carry the active molecules or the effective groups which it contains into the body fluids and deliver them to various body compartments. In particular, the functionalized carbon nanotubes can deliver the active molecule to blood, lymph or mucosae.

The invention also relates to the use of a functionalized carbon nanotube of the invention, for the delivery of drugs, in particular for the intracellular delivery of drugs.

It has been found, in a very unexpected manner, that the functionalized carbon nanotubes according to the invention can penetrate into cells, thus carrying into the cellular compartment the active molecule or effective group to which it is covalently bound.

Advantageously, the active molecule and effective group contained in the functionalized carbon nanotube can be cleaved from the rest of the functionalized carbon nanotube and liberated in the cytoplasm of cells into which the functionalized nanotube has penetrated. For this purpose the use of linker groups sensitive to physiological conditions is advantageous. Such linkers in particular comprise linkers of the following formulae:

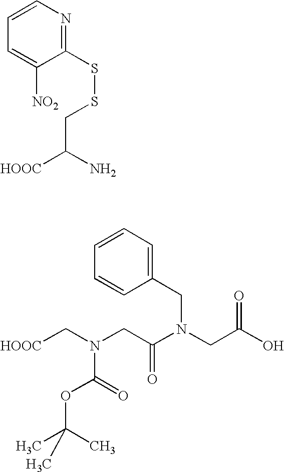

The functionalized carbon nanotubes of the invention can also be used for the preparation of an immunogenic composition intended to provide an immunological protection to the individual to whom it has been administered.

Advantageously, the nanotube by itself is not immunogenic, i.e. no antibodies directed against the carbon wall of the nanotube can be detected in the serum of individuals or mice, to which a functionalized nanotube has been administered.

This property can be in particular illustrated by the following experiment:

BALB/c mice are immunized with an amino functionalized carbon nanotube in the presence of ovalbumin (OVA) and complete Freund's adjuvant (one injection and a boost injection after 3 weeks). Serum samples are then collected and an ELISA test performed against the functionalized carbon nanotube adsorbed on the plate.

Carbon nanotubes do not induce the production of antibodies directed against the carbon nanotube in itself, said antibodies being liable to interfere with the immune response to the epitope carried by the functionalized carbon nanotube.

The functionalized carbon nanotubes of the invention can be used for the preparation of a medicament intended for the treatment or the prophylaxis of cancer, autoimmune or infectious diseases.

The diseases, which can be treated are for instance solid tumors, such as prostate tumors, melanoma, autoimmune diseases, such as Systemic Lupus Erythematosus (SLE), rheumatoid poly-arthritis (RP), diabetes, HIV, hepatitis, malaria or tuberculosis.

The functionalized carbon nanotubes of the invention can be used for the preparation of functionalized surfaces such as plastic or glass surfaces.

These surfaces can be functionalized by simple adsorption of the functionalized carbon nanotubes of the invention. Adsorption mainly occurs through the establishment of hydrophobic interactions between the surface carbon of the carbon nanotubes and the glass or plastic surface. In particular the functionalized carbon nanotube of the invention can be adsorbed to plastic ELISA plate wells.

Optionally the functionalized carbon nanotubes of the invention can be oxydized to generate carboxyl function at the extremities of the carbon nanotubes, said carboxyl functions allowing covalent linkage of the functionalized carbon nanotubes to plastic or glass surfaces, provided that said surfaces present group capable of forming a covalent bond with the carboxyl function.

The functionalized carbon nanotubes of the invention can also be used for the preparation of electrochemical biosensors.

### BRIEF DESCRIPTION OF THE DRAWINGS

FIG. 1A and FIG. 1B

FIGS. 1A and 1B respectively represent the transmission electron microscopy images of carbon nanotubes functionalized with peptide KGYYG and with peptide Acetyl-CGSGVRGDFGSLAPRVARQL. In FIG. 1A the horizontal bar corresponds to a length of 400 nm and in FIG. 1B the horizontal bar corresponds to a length of 100 nm.

FIG. 2A and FIG. 2B

FIGS. 2A and 2B respectively represent partial bidimensional ¹H NMR TOCSY spectra of carbon nanotubes functionalized with peptide KGYYG and with peptide Acetyl-CGSGVRGDFGSLAPRVARQL in H₂O/t-BuOH-d₉ 9:1 solution. TEG stands for triethylene glycol. The horizontal and vertical axes represent chemical shifts in ppm (parts per million).

In FIG. 2A peptide residues are numbered from K1 to G5. The bidimensional spectrum has been recorded decoupling ¹⁵N heteronucleus.

In FIG. 2B peptide residues are numbered from C1 to L20.

FIG. 3

FIG. 3 represents a fluorescence microscopy picture of 3T3 murine cells which have been incubated during 40 minutes with FITC -functionalized carbon nanotubes of the invention.

FIG. 4

FIG. 4 represents Biacore sensorgrams obtained by allowing analytes to react on a monoclonal anti-peptide antibody. The vertical axis corresponds to the response expressed in resonance unit (RU) (1000 RU=1 ng/mm² of analyte) plotted against the time expressed in seconds (horizontal axis).

The association phase took 4 min, the dissociation phase 5 min. Curve (a) represents the response with the Acetyl-CGSGVRGDFGSLAPRVARQL peptide functionalized carbon nanotube (6 μM), curve (b) represents the response with free Acetyl-CGSGVRGDFGSLAPRVARQL peptide (5 μM) and curve (c) represents the acetylated functionalized carbon nanotube used at the same concentration as the peptide functionalized carbon nanotube.

FIG. 5A and FIG. 5B

FIGS. 5A and 5B represent the recognition of the peptide Acetyl-CGSGVRGDFGSLAPRVARQL displayed onto carbon nanotubes by polyclonal (FIG. 5A) and monoclonal 21×27 (FIG. 5B) anti-peptide antibodies (as defined in Example 9). Data are represented as absorbance values measured at 450 nm (vertical axis) versus antibody dilution (horizontal axis) for ELISA plates coated with different peptide preparations:

ELISA plates were coated with 5 μg/ml of free peptide (hyphened line), or 5 μg/ml of peptide functionalized carbon nanotubes (calculated on the basis of peptide loading on the nanotube side-walls) (continuous line), or a control functionalized carbon nanotube used at the same concentration (hyphened line with short and long stretches) in carbonate/bicarbonate buffer.

FIG. 6

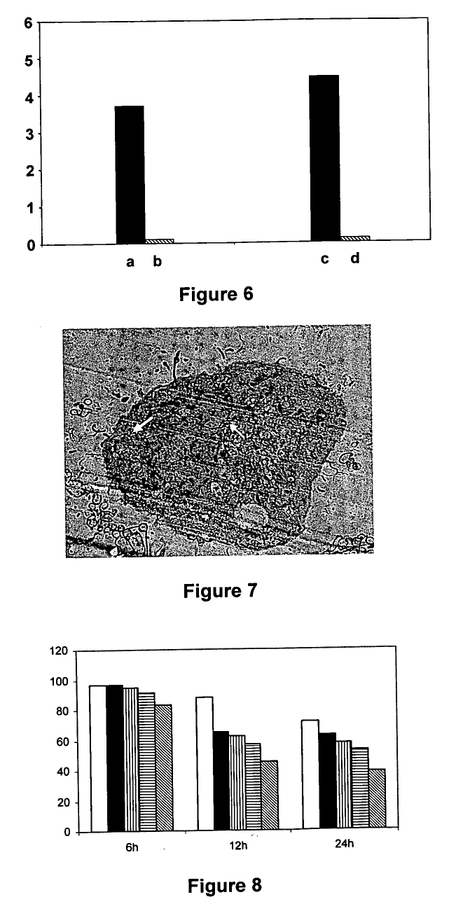

FIG. 6 represents the quantity of antibodies, expressed as the decimal logarithm of the antibody titer (vertical axis), present in serum samples of BALB/c mice immunized with:
free Acetyl-GSGVRGDFGSLAPRVARQL peptide, said antibodies being directed against (horizontal axis) the peptide conjugated to BSA (black bar, a) or a maleimide functionalized carbon nanotube (hatched bar, b),
Acetyl-CGSGVRGDFGSLAPRVARQL peptide functionalized carbon nanotubes, said antibodies being directed against (horizontal axis) the peptide conjugated to BSA (black bar, c) or against a maleimide functionalized carbon nanotube (hatched bar, d).

FIG. 7

FIG. 7 represents the transmission electron microscopy (TEM) image of functionalized MWNTs according to the invention inside a cell (white arrows).

FIG. 8

FIG. 8 represents the percentage of living cells after treatment with functionalized SWNT at (from left to right) 0 (white bars), 0.001 (black bars), 0.01 (vertically hatched bars), 0.1 (horizontally hatched bars), and 1 mg/ml (obliquely hatched bars) for 6 h, 12 h and 24 h.

### EXAMPLES

### Example 1

### Preparation of a Reactive Functionalized Carbon Nanotube

The compound of the following formula (A) was prepared according to the following protocol:

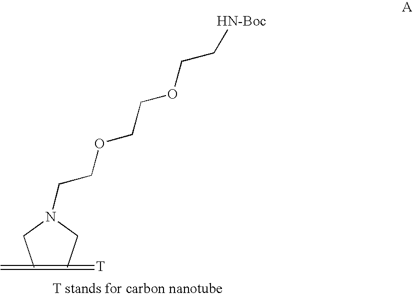

100 mg of single-walled carbon nanotubes (SWNTs) (Carbon Nanotechnologies Inc., USA) were suspended in 300 ml of dimethylformamide (DMF). The mixture was heated at 130° C. and 300 mg of tert-butoxycarbonyl (Boc) N-protected amino acid (B) were added together with 150 mg of paraformaldehyde at the beginning and then every 24 hours.

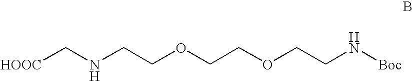

The mixture was heated for 96 hours. After separation of the unreacted material by filtration, followed by evaporation of the DMF, the resulting residue was diluted with 100 ml of dichoromethane (DCM) and washed with water (1×50 ml). The organic phase was dried over Na₂SO₄, filtered and evaporated under vacuum. The residue was dissolved in 1 ml of dichloromethane and isolated by centrifugation upon precipitation with diethyl ether. The solid was subsequently washed 5 times with ether. The yield, based on the amount of starting SWNTs was about 10%. This yield can reach 30-40% if part of the material remained in the water phase after the first extraction is recovered. The final material resulted soluble in most common organic solvents such as acetone, chloroform, dichloromethane, toluene, methanol and ethanol. They are also partially soluble in water.

The protected functionalized nanotube thus obtained was then submitted to deprotection. To a solution of SWNTs of molecular structure (A) in dichloromethane (100 mg in 20 ml), gaseous HCl was bubbled along 1 hour to remove the tert-butoxycarbonyl protecting group (Boc) at the chain-end. The corresponding SWNT ammonium chloride salt precipitates during the acid treatment. After removal of the solvent under vacuum, the brown solid was dissolved in 1 ml of methanol and precipitated with diethyl ether. The residue was washed 5 times with diethyl ether to obtain the product of formula (C). The yield was quantitative. The loading of carbon nanotubes was calculated with a quantitative Kaiser test (Sarin, V. K. et al. Anal. Biochem. (1981) 117:147-157) and correspond to about 0.5 mmol/g. Deprotected SWNTs possess a remarkably high solubility in water 20 mg of product give a stable solution in 1 ml of water for more than a month. The purity of the material was determined by transmission electron microscopy (TEM) analysis; briefly, carbon nanotube derivatives were suspended in diethyl ether and sonicated for 15 min, 30 μL were then deposited on special grids for TEM analysis (Formvar carbon supported film on 200/400 mesh copper grids (Electron Microscopy Sciences, Fort Washington, USA)); the analysis is performed on a TEM H600 (Hitachi Europe Ltd., Maidenhead, UK) at 110 kV at different magnifications.

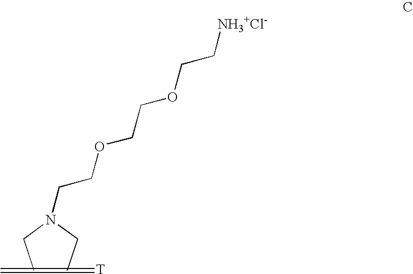

Alternatively, the compound of the following formula (A′) was prepared according to the following protocol:

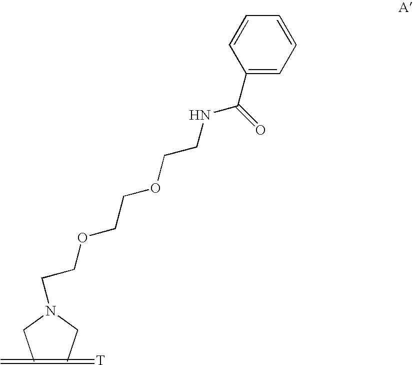

100 mg of single-walled carbon nanotubes (SWNTs) (Carbon Nanotechnologies Inc., USA) were suspended in 300 ml of dimethylformamide (DMF). The mixture was heated at 130° C. and 300 mg of benzoyl (Bz) N-protected amino acid (B′) were added together with 150 mg of paraformaldehyde at the beginning and then every 24 hours.

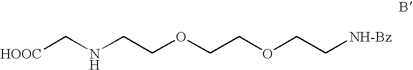

The mixture was heated for 96 hours. After separation of the unreacted material by filtration, followed by evaporation of the DMF, the resulting residue was diluted with 100 ml of dichloromethane (DCM) and washed with water (1×50 ml). The organic phase was dried over Na₂SO₄, filtered and evaporated under vacuum. The residue was dissolved in 1 ml of dichloromethane and isolated by centrifugation upon precipitation with diethyl ether. The solid was subsequently washed 5 times with ether. The yield, based on the amount of starting SWNTs was about 10%. This yield can reach 30-40% if part of the material remained in the water phase after the first extraction is recovered. The final material resulted soluble in most common organic solvents such as acetone, chloroform, dichloromethane, toluene, methanol and ethanol. They are also partially soluble in water.

The protected functionalized nanotube thus obtained was then submitted to deprotection. A solution of SWNTs of molecular structure (A′) was treated in 6 N HCl for 24 hours to remove the benzoyl (Bz) protecting group at the chain-end. After removal of the solvent under vacuum, the brown solid was dissolved in 1 ml of methanol and precipitated with diethyl ether. The residue was washed 5 times with diethyl ether to obtain the product of formula (C). The yield was quantitative. The loading of carbon nanotubes was calculated with a quantitative Kaiser test and correspond to about 0.5 mmol/g. The purity of the material was determined by transmission electron microscopy (TEM) as described above.

### Example 2

### Amino Acid Functionalization of Deprotected Reactive Carbon Nanotubes

9 mg of Fmoc-Gly-OH (two-fold excess) were activated with 4 mg of N-hydroxybenzotriazole (HOBt) and 5 μl of diisopropylcarbodiimide (DIC) in 1 ml of a 1:1 mixture of DMF/DCM for 15 min and added to a suspension of 10 mg of deprotected carbon nanotubes (C) in DCM, previously neutralized with 24 μl diisopropylethylamine (DIEA). After stirring at room temperature for 2 hours, the coupling reaction was terminated (negative Kaiser test) and the solvent was completely evaporated. The crude material was dissolved in 2 ml of DCM and reprecipitated 7 times by addition of diethyl ether. The yield was quantitative. Carbon nanotubes functionalized with the protected amino acid (see formula D) were characterized by TEM, as described in Example 1, and NMR spectroscopy.

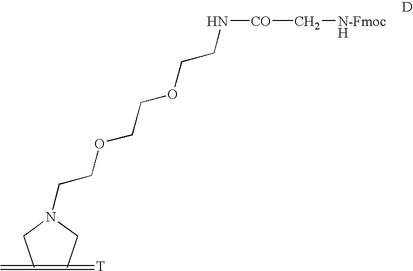

The Fmoc protecting group was removed by treatment with 25% piperidine in DMF for 10 minutes (twice). After evaporation of the solvent, the crude material was dissolved in DCM and reprecipitated by addition of diethyl ether to obtain the compound of molecular structure (E):

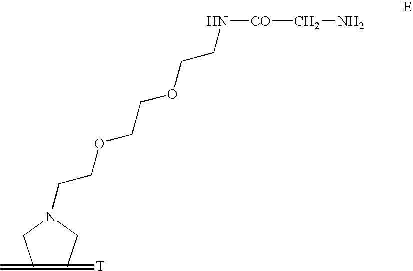

### Example 3

### Acetyl Functionalization of Deprotected Reactive Carbon Nanotubes

3 mg, of functionalized carbon nanotube of formula (C) were suspended in 500 μl of DCM and neutralized with 10 μl of DIEA. 1 ml of 25% acetic anhydride in DCM was added. The reaction was stirred for 30 minutes at room temperature. The solvent was then evaporated and the crude material obtained dissolved in methanol, it was then precipitated 3 times by addition of diethyl ether and the solid was lyophilized in water to obtain the product of formula (K). The yield was quantitative. The product of formula (K) was characterized by TEM, as described in Example 1

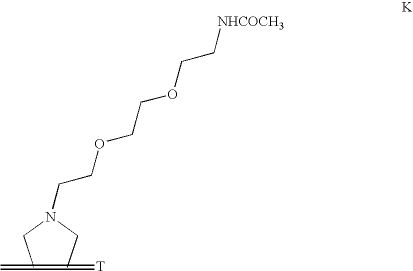

### Example 4

### FITC Functionalization of Deprotected Reactive Carbon Nanotubes

4 mg of the functionalized carbon nanotube of formula (C) were suspended in 150 μl of DMF and neutralized with 2 μl of DIEA. A solution of 5 mg of FITC (fluoresceine isothiocyanate, isomer 1) in 50 μl of DMF was added and the reaction stirred overnight at room temperature under argon. The solvent was evaporated, the crude material was then dissolved in methanol and reprecipitated 10 times by addition of diethyl ether, to obtain the product of formula (L). The yield was quantitative. The product of formula (L) was characterized by TEM, as described in Example 1, and by florescence microscopy.

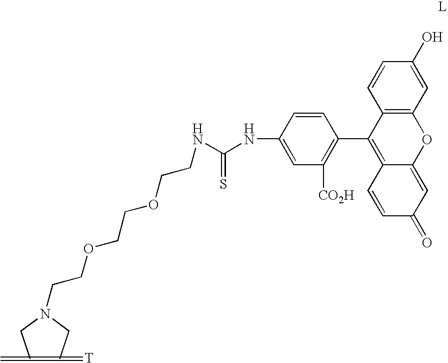

### Example 5

### Peptide Condensation on a Reactive Functionalized Carbon Nanotube

Deprotected carbon nanotubes (10 mg, corresponding to 4 μmol based on the loading calculated by the quantitative Kaiser test) of molecular structure (C) suspended in 2 ml of DMF were neutralized in DIEA (80 μL, 46 μmol). A solution of fully-protected peptide (Boc-Lys(Boc)-Gly-Tyr(tBu)-Tyr(tBu)-Gly-OH) (61.3 mg, 6.7 μmol) in 2 ml of DIME was activated with O-(7-aza-N-hydroxybenzotriazol-1-yl)-1,1,3,3-tetramethyluronium hexafluorophosphate (HATU) (29.7 mg, 7.8 μmol) for 10 min and subsequently added to the reactive functionalized carbon nanotube. The mixture was stirred for 2 hours. The solvent was evaporated and the crude product was solubilized in methanol and reprecipitated by addition of diethyl ether. After centrifugation, the precipitate was dried under vacuum. The fully-protected peptide-carbon nanotube conjugate, of formula (F), was solubilized in 0.5 ml of methanol and treated with 1 ml of a 4 M HCl solution in dioxane.

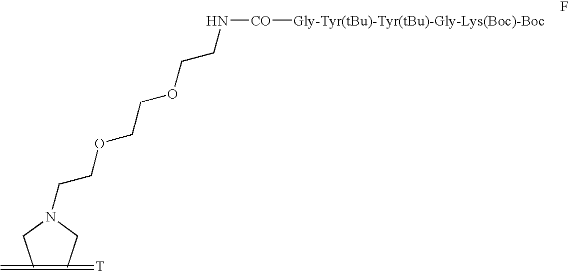

After stirring for 1 hour, the product of formula (G) was obtained by precipitation in cold diethyl ether. The yield was quantitative.

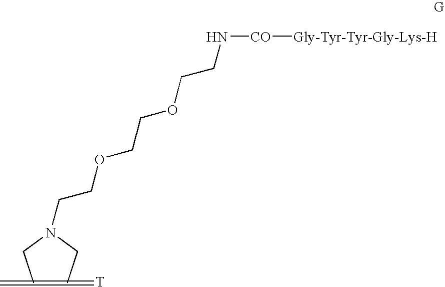

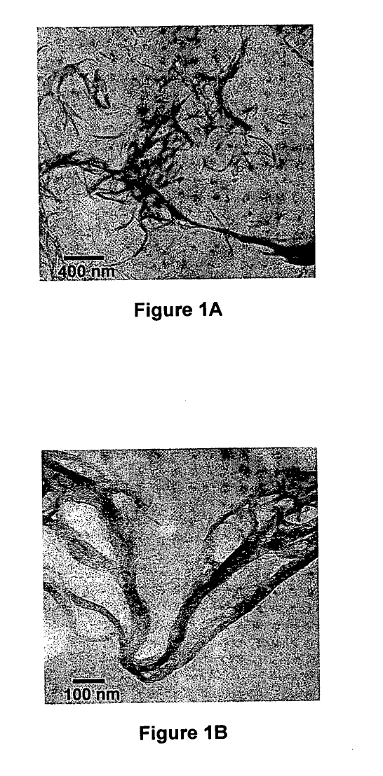

Carbon nanotubes functionalized with the peptide were first characterized by TEM, as described in Example 1, (FIG. 1A), which allowed the visualization of bundles of carbon nanotubes of different diameters, ranging form 8 to 53 nm.

The carbon nanotubes functionalized with KGYYG were also studied by NMR spectroscopy either with the fully-protected peptide or with the N-terminus and side-chain free peptide in CD₃CN and H₂O/tBuOH-d₉ solution, respectively. Briefly, the identification of amino acid spin systems and sequential assignment were made using a combination of TOCSY (Rucker S. P. et al. J. Mol. Phys. (1989) 63:509-517), NOESY (Jeener J. et al. J. Chem. Phys. (1979) 71:45464553), ROESY (Desvaux H., J. Magn. Res. A (1995) 113:47-52) and HMQC (Bax A. et al. J. Magn. Res. (1983) 55:301-335) experiments. 1D and 2D NMR spectra were recorded on an ARX 500 MHz spectrometer (Brulcer, Wissenbourg, France). The sample was dissolved in CD₃CN or H₂O/tBuOH-d₉ 9:1. The spectra were acquired at a temperature of 300 K and referenced to the peak of the solvent. WATERGATE pulse sequence was applied for the suppression of water signal (Piotto M. et al. J. Biomol. NMR (1992) 2:661-665).

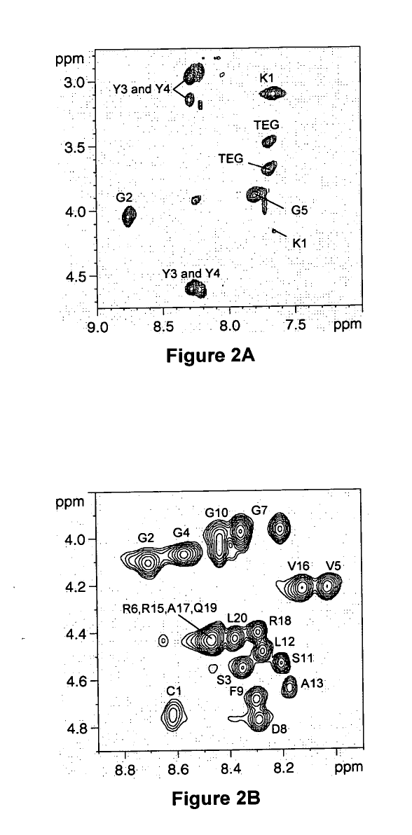

Due to the presence of a N¹⁵-labelled Gly at the C-terminal part, homo- and heteronuclear 2D NMR spectra were recorded. A broad correlation peak in the decoupled ¹⁵N—¹H spectrum of the fully-protected compound of formula (F), with the maximum peak height measured at 119.6/7.40 ppm, was indicative of a homogenous distribution of peptide around the nanotube side-wall. A series of bi-dimensional experiments permitted then to assign all the resonances of the peptide moiety. A decrease and a broadening of the signal intensities were observed for the amino acid residues approaching the aromatic tube walls. In the case of deprotected peptide-carbon nanotube (G), all the residue signals were attributed in a H₂O/tBuOH-d₉ 9:1 solution (FIG. 2A). All the expected sequential αHᵢ—NHᵢ₊₁ cross-peaks were present in the ROESY spectrum, but most importantly a spatial correlation between the αH of glycine at position 5 and the amide proton of the oligoethylene glycol chain confirmed the covalent bond between the peptide and the carbon nanotubes.

### Example 6

### Chemoselective Ligation of a Peptide on a Reactive Functionalized Carbon Nanotube

Deprotected carbon nanotubes (7.0 mg, 2.8 μmol) of molecular structure (C) suspended in 2 ml of DMF were neutralized with DIEA (15 μL, 8.5 μmol). N-Succinimidyl 3-maleimidopropionate (12 mg, 45 μmol) dissolved in 2 ml of DMF was added and the reaction was stirred for 6 hours at room temperature. The excess of N-succinimidyl 3-maleimidopropionate was eliminated overnight by adding 50 mg of PEGA-NH₂ resin (Novabiochem, Laufelfingen, Switzerland). The resin was eliminated by filtration and the solvent was evaporated. The product of formula (H) was dissolved in methanol and reprecipitated five times with diethyl ether.

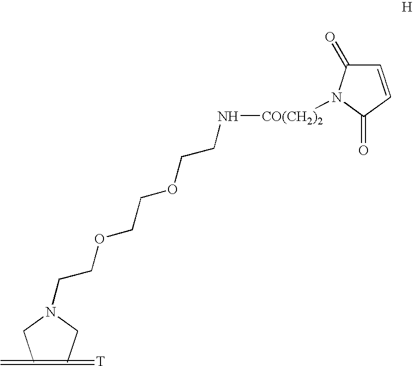

To a solution of the carbon nanotubes functionalized with the succinimidyl group of formula (H) (4.0 mg, 1.6 μmol) in 1.5 ml of water, a deprotected peptide, acetylated at the N-terminus (Acetyl-CGSGVRGDFGSLAPRVARQL) (4.0 mg, 1.91 μmol) was added, in order to covalently link the thiol group of the Cys in position 1 to the maleimido group. This peptide, hereinafter designated as Ac-Cys-FMDV, is a B-cell epitope which represents amino acids 141-159 from the coat protein VP1 of the foot-and-mouth disease virus. The reaction was stirred for 6 hours at room temperature, and 70 mg of PEGA-NH₂, resin previously derivatized with compound N-succinimidyl 3-maleimidopropionate was added in order to eliminate the excess of peptide overnight. The resin was eliminated by filtration and the water solution lyophilized. The yield was 79%. Carbon nanotubes functionalized with the peptide, of formula (J), represented hereafter, were characterized by TEM (FIG. 1B) as described in Example 1, except that the functionalized peptide nanotube was solubilized in methanol.

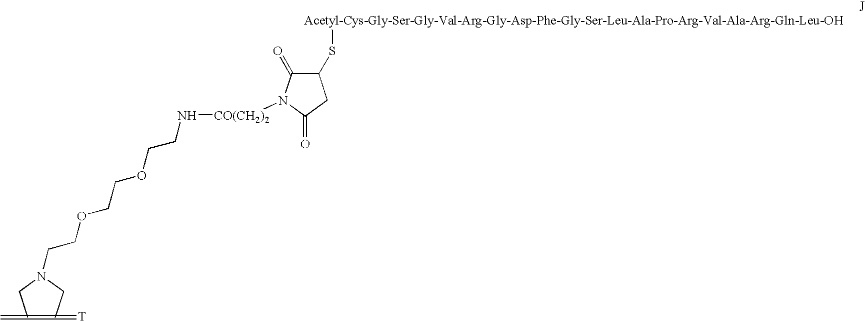

The peptide functionalized carbon nanotube (J) was also studied by NMR spectroscopy, as described in Example 5. Briefly, a series of TOCSY, NOESY and ROESY spectra were acquired in a H₂O/tBuOH-d₉ 9:1 solution, which allowed to fully assign the twenty amino acid residues (FIG. 2B). The chemical shift dispersion, and the intensity and position of NOEs (nuclear Overhauser effect) were very similar (except for some residues at both sequence termini) to those of the same peptide previously studied in aqueous solution free (Petit M. C. et al. J. Biol. Chem. (1999) 274:3686-3692), or bound to POEPOP resin (Furrer J. et al. J. Am. Chem. Soc. (2001) 123:4130-4138). This suggests that the peptide displays the same conformational behaviour when it is free or linked to the carbon nanotubes.

The following peptides can also be added to the functionalized carbon nanotube of formula (H) according to the above described method:

1. QRMHLRQYELLC

2. CQRMHLRQYELL

3. K(FITC)QRMHLRQYELLC

4. CRIHMVYpSKRSGKPRGYAFIEY

5. CVGFPVTPQVPLRPMTYKAAVDLSHFLKEKGGL
N.B.: in peptide 3, FITC is linked to the εNH₂ of K in position 1; in peptide 4, pS stands for phosphoserine.

Alternatively, the C in position 1 of peptide 5 can be replaced by a 3-nitro-2-pyridylsulfenyl (NPys) protected C to form the following peptide:
C(NPys)VGFPVTPQVPLRPMTYKAACDLSHFLKEKGGL
   which can be linked through its thiol group to a cysteine functionalized nanotube, prepared according to Example 2, via a disulfide exchange reaction, to form the compound of molecular structure (M):

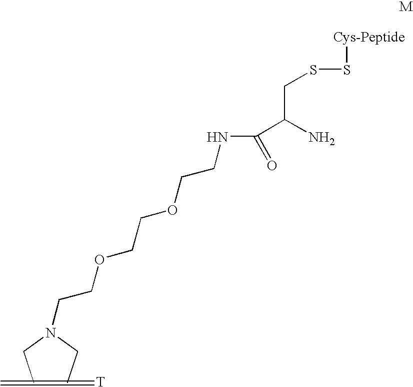

Briefly, two-fold excess of N- and S-protected cysteine were activated with a coupling reagent (HOBt/DIC) in DMF/DCM and added to a suspension of deprotected carbon nanotube (C) in DCM, previously neutralized with DIEA. After stirring for 2 h, the solvent was evaporated, the crude material dissolved in DCM and reprecipitated several time by addition of diethyl ether. The N- and S-protecting groups were removed and the free cysteine functionalized carbon nanotube was dissolved in a buffer solution at pH 6.5 followed by addition of 1.5-fold excess of peptide C(NPys)VGFPVTPQVPLRPMTKAAVDLSHFLKEKGGL. The mixture was stirred for 6 h at room temperature and the excess of peptide eliminated using a scavenger resin according to the above described procedure. The peptide-carbon nanotube of formula (M) was recovered after filtration of the resin and lyophilization.

The same functionalization has been performed with peptides 1-4 as well as with a scrambled peptide 4 (CYVSRYFGpSAIRHEPKMKIYRG).

### Example 7

### DTPA Functionalization of Deprotected Reactive Carbon Nanotubes

590 μg of DTPA (diethylenetriaminepentaacetic acid dianhydride) (0.6 equiv) were added to a solution of 5.5 mg (loading 0.55 mmol/g) of deprotected carbon nanotubes (C) in 1 ml of dimethylsulfoxide, previously neutralized with 1 μl diisopropylethylamine (DIEA). After stirring at room temperature overnight (16 hours), the solution was diluted with water and lyophilized. Lyophilization was repeated three times. The yield was quantitative. The amount of remaining amino functions was calculated with the quantitative Kaiser test and resulted around 50% of the initial loading. DTPA functionalized carbon nanotubes (N) were characterized by TEM, as described in Example 1.

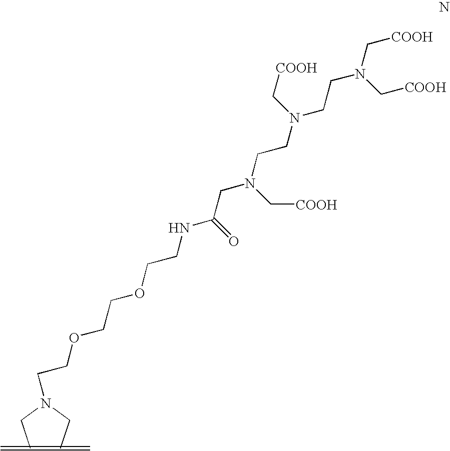

DTPA-functionalized carbon nanotubes can be complexed with [¹¹¹In]³⁺in the following way. [¹¹¹In]citrate is mixed with compound (N) in water at pH 7-8. After 24 hours the excess of [¹¹¹In]³⁺is removed overnight by adding PEGA-NH₂ resin (Novabiochem) previously functionalized with DTPA, which is removed by filtration. The solution is lyophilized until use in the animal biodistribution experiments.

### Example 8

### Step-by-Step Peptide Synthesis Using a Carbon Nanotube Support

Fmoc-Xaa-OH or Boc-Xaa-OH (Xaa can be any possible amino acid) (three-fold excess) was activated with a coupling reagent (for example a mixture of HOBt/BOP/DIEA) in DMF for 15 min and added to a suspension of the reactive functionalized nanotube of formula (C) or of a carbon nanotube functionalized with a reactive amino group in DCM, previously neutralized with DIEA. After stirring at room temperature for 2 hours, the carbon nanotubes derivatized with the first amino acid were precipitated by addition of diethyl ether. After centrifugation, the crude product was solubilized again in methanol or dichloromethane and reprecipitated by addition diethyl ether. This procedure was repeated 5 times. The N-protecting group Fmoc or Boc was cleaved by treatment with a solution of 25% piperidine in DMF or TFA, respectively, and the amino acid functionalized carbon nanotube was precipitated with diethyl ether. After centrifugation, the precipitate was solubilized again in methanol or dichloromethane and reprecipitated by addition diethyl ether. This procedure was repeated 5 times. The following amino acids were coupled using the same, conditions as those used for the coupling of the first amino acid. At the end of the amino acid sequence, the side-chain protecting groups were cleaved and the carbon nanotubes functionalized with the peptide are characterized by TEM microscopy and amino acid analysis.

By using a suitable linker between the amino functionalized carbon nanotube and the peptide, it is possible to remove the peptide from the carbon nanotube and characterize it by reverse phase HPLC (high performance liquid chromatography) and mass spectrometry.

### Example 9

### Preparation of bis-FMDV peptide-carbon nanotube conjugate

Amino-functionalized carbon nanotube (C) (5.0 mg, 2.5 μmol) was dissolved in 1 ml of dichloromethane and neutralized with DIEA (4 μl, 22.5 μmol). A solution of Boc-Lys(Boc)-OH (2.6 mg, 7.5 μmol) in 1 ml of DMF was activated with DIC (diisopropylcarbodiimide) (1.2 μl, 7.5 μmol) and HOBt (1-hydroxybenzotriazole) (1.2 mg, 7.8 μmol) for 10 min and subsequently added to carbon nanotube solution. The mixture was stirred for 3 hours. The solvent was evaporated and the product of formula (O) obtained, was precipitated several times from methanol with diethyl ether and dried under vacuum. The disappearance of the excess of Boc-Lys(Boc)-OH was followed by thin-layer chromatography (dichloromethane/methanol 8:2).

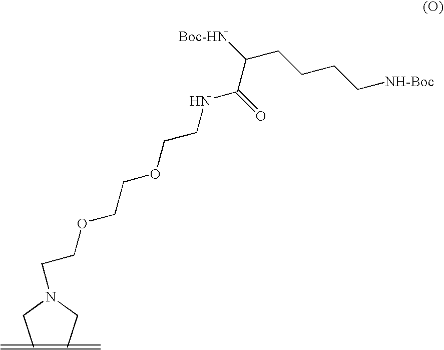

The Boc protecting group was removed from the functionalized carbon nanotubes of formula (O) by treatment with 2 ml of trifluoroacetic acid (TFA) overnight to yield a compound of formula (P). The product was recovered as a brown solid after several precipitations in cold diethyl ether.

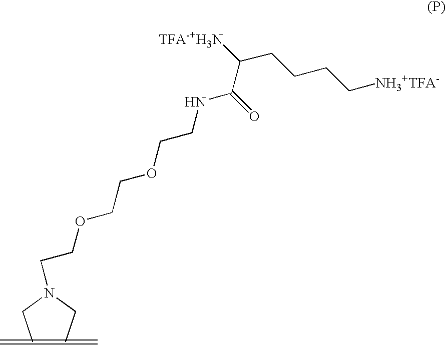

The deprotected carbon nanotube was dissolved in 1 ml of DMF and neutralized with DIEA (30.0 μl, 169.5 μmol). N-Succinimidyl 3-maleimidopropionate (13.0 mg, 48.8 μmol) dissolved in 1 ml of DMF was added and the reaction mixture stirred at room temperature overnight. The excess of maleimido derivative was removed overnight by adding 70 mg of PEGA-NH₂ resin (Novabiochem), which was removed by filtration, and the solvent was evaporated. The product (Q) obtained was dissolved in methanol and precipitated several times with cold diethyl ether.

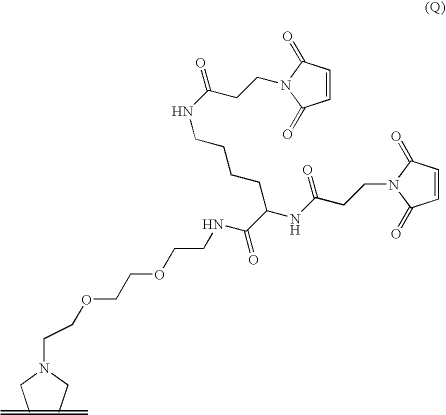

To a solution of the carbon nanotubes functionalized with the maleimido group (Q) (4.0 mg, 2.0 μmol) in 2 mL of water, Ac-Cys-¹⁴¹GSGVRGDFGSLAPRVARQL¹⁵⁹ (Ac-Cys-FMDV peptide) (11.0 mg, 5.2 μmol) was added. The reaction was stirred for 9 hours at room temperature and 70 mg of PEGA-NH₂ resin previously derivatized with N-succinimidyl 3-maleidopropionate were added to remove the excess of peptide after 5 hours. The resin was removed by filtration and the solvent was lyophilized to provide carbon nanotube (bis-FMDV-peptide conjugate) of formula (R). The removal of the free FMDV peptide after the addition of the scavenger resin was monitored by RP-HPLC on a Macherey-Nagel C₁₈ column using a linear gradient of A: 0.1% TFA in water and B: 0.08% TFA in acetonitrile, 5-65% B in 20 min at 1.2 mL/min flow rate.

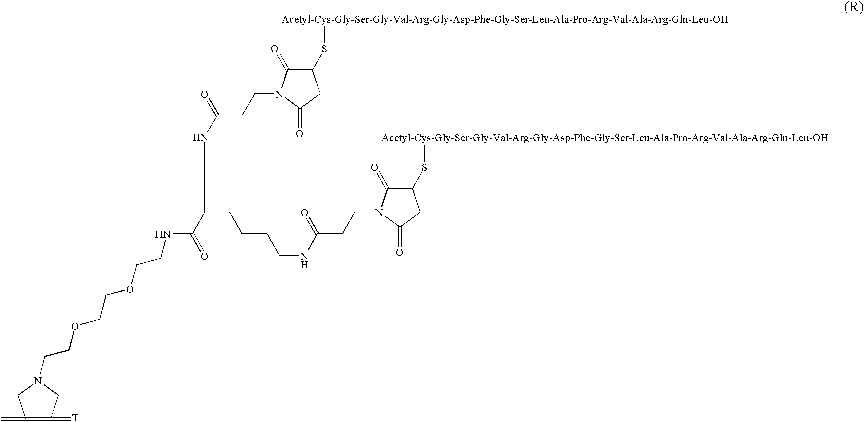

### Example 10

### Preparation of Carbon Nanotube Conjugate with Two Different Peptides

Amino-functionalized carbon nanotubes (C) are dissolved in dichloromethane and neutralized with DIEA (diisopropylethylamine). A solution of Fmoc-Lys(Boc)-OH in DMF is activated with DIC (diisopropylcarbodiimide) and HOBt (1-hydroxybenzotriazole) for 10 min and subsequently added to carbon nanotube solution. The mixture is stirred for 3 hours. The solvent is evaporated and the obtained product (S) precipitated several times from methanol with diethyl ether and dried under vacuum. The disappearance of the excess of Fmoc-Lys(Boc)-OH is followed by thin-layer chromatography.

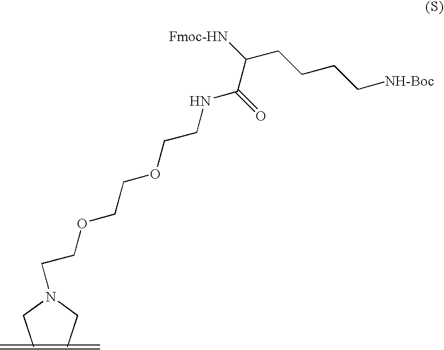

The Boc protecting group is removed from the functionalized carbon nanotubes by treatment with trifluoroacetic acid (TFA) for two hours. The product (U) is recovered as a brown solid after several precipitations in cold diethyl ether.

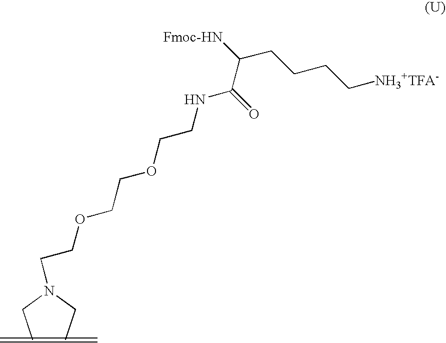

The mono-deprotected carbon nanotube (U) is dissolved in DMF and neutralized with DIEA. A solution of fully-protected peptide ⁸³⁰QYIKANSKFIGITE⁸⁴³ in DMF is activated with O-(7-aza-N-hydroxybenzotriazol-1-yl)-1,1,3,3-tetramethyluronium hexafluorophosphate (HATU) for 10 min and subsequently added to carbon nanotube. The mixture is stirred for 2 hours. The solvent is evaporated and the crude product is solubilized in methanol and reprecipitated by addition of diethyl ether. After centrifugation it is dried under vacuum.

The Fmoc protecting group is then removed from the functionalized carbon nanotubes by treatment with 25% piperidine in DMF for 15 minutes (twice). N-Succinimidyl 3-maleimidopropionate (5 equiv) dissolved in 1 ml of DMF is added and the reaction mixture stirred at room temperature overnight. The excess of maleimido derivative is removed overnight by adding 70 mg, of PEGA-NH₂ resin (Novabiochem), which is removed by filtration, and the solvent is evaporated. The product is dissolved in methanol and precipitated several times with cold diethyl ether.

To a solution of the carbon nanotubes functionalized with the maleimido group in water, Cc-Cys-¹⁴¹⁻GSGRGDFGSLAPRVARQL¹⁵⁹ (Ac-Cys-FMDV peptide) is added. The reaction is stirred for 9 hours at room temperature and 70 mg of PEGA-NH₂ resin previously derivatized with N-succinimidyl 3-maleimidopropionate are added to remove the excess of peptide after 5 hours. The resin is removed by filtration and the solvent is lyophilized to provide carbon nanotube conjugate with two different peptides, one still protected. The partially fully-protected peptide-carbon nanotube conjugate is treated with 1 ml of a 4 M HCl solution in dioxane. After stirring 1 hour, the product (V) is obtained by precipitation in cold diethyl ether. Carbon nanotubes functionalized with the two different peptides are characterized by TEM microscopic and NMR spectroscopy.

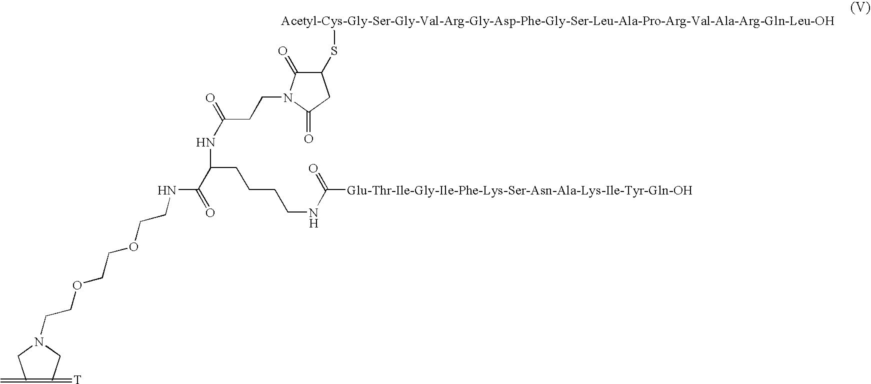

### Example 11

### Preparation of a Reactive Bis-Functionalized Carbon Nanotube with Two Different

Peptides

The compound of the following formula (A′) can be prepared according to the following protocol:

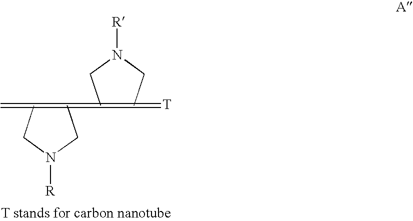

100 mg of single-walled carbon nanotubes (SWNTs) (Carbon Nanotechnologies Inc., USA) are suspended in 300 ml of dimethylformamide (AMF). The mixture is heated at 130° C. and 150 mg of tert-butoxycarbonyl (Boc) N-protected amino acid (B) and 150 mg phathalimide (Pht) N-protected amino acid (B′) are added together with 150 mg of paraformaldehyde at the beginning and then every 24 hours.

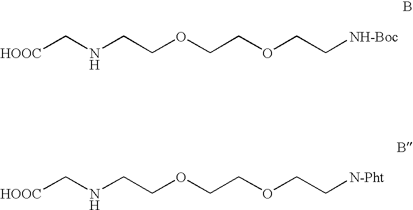

The mixture is heated for 96 hours. After separation of the unreacted material by filtration, followed by evaporation of the DMF, the resulting residue is diluted with 100 ml of dichloromethane (DCM) and washed with water (1×50 ml). The organic phase is dried over Na₂SO₄, filtered and evaporated under vacuum. The residue is dissolved in 1 ml of dichloromethane and isolated by centrifugation upon precipitation with diethyl ether. The solid is subsequently washed 5 times with ether.

The orthogonally protected bis-functionalized nanotube thus obtained is then submitted to selective deprotection. To a solution of SWNTs of molecular structure (A′) in dichloromethane (100 mg in 20 ml), gaseous HCl is bubbled along 1 hour to remove the tert-butoxycarbonyl protecting group (Boc) at the chain-end. The corresponding SWNT ammonium chloride salt precipitates during the acid treatment. After removal of the solvent under vacuum, the brown solid is dissolved in 1 ml of methanol and precipitated with diethyl ether. The residue is washed 5 times with diethyl ether to obtain the product of formula (C′). The loading of carbon nanotubes is calculated with a quantitative Kaiser test (Sarin, V. K. et al. Anal. Biochem. (1981) 117:147-157). The purity of the material is determined by transmission electron microscopy (TEM) analysis; briefly, carbon nanotube derivatives are suspended in diethyl ether and sonicated for 15 min, 30 μL are then deposited on special grids for TEM analysis [Formvar carbon supported film on 200/400 mesh copper grids (Electron Microscopy Sciences, Fort Washington, USA)]; the analysis is performed on a TEM H600 (Hitachi Europe Ltd., Maidenhead, UK) at 110 kV at different magnifications.

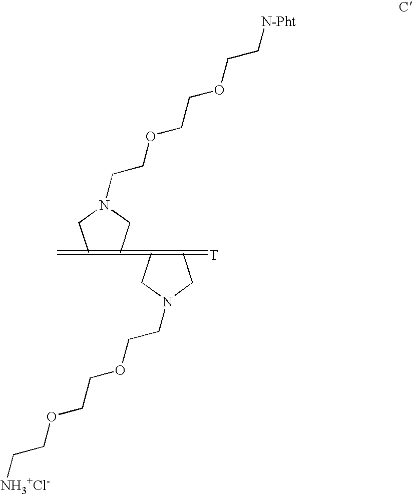

The mono-deprotected carbon nanotube is dissolved in DMF and neutralized with DIEA. A solution of fully-protected peptide ⁸³⁰QYIKANSKFIGITE⁸⁴³ in DMF is activated with O-(7-aza-N-hydroxybenzotriazol-1-yl)-1,1,3,3-tetramethyluronium hexafluorophosphate (HATU) for 10 nm in and subsequently added to carbon nanotube. The mixture is stirred for 2 hours. The solvent is evaporated and the crude product is solubilized in methanol and reprecipitated by addition of diethyl ether. After centrifugation it is dried under vacuum.

Deprotection of Pht is performed by treating the peptide-functionalized carbon nanotube with hydrazine in ethanol (0.5 ml) for 12 hours to liberate the second amino function. N-Succinimidyl 3-maleimidopropionate dissolved in 1 ml of DMF is added and the reaction mixture stirred at room temperature overnight. The excess of maleimido derivative is removed overnight by adding 70 mg of PEGA-NH₂ resin (Novabiochem), which is removed by filtration, and the solvent is evaporated. The product is dissolved in methanol and precipitated several times with cold diethyl ether.

To a solution of the carbon nanotubes functionalized with the maleimido group in water, Ac-Cys-¹⁴¹GSGVRGDFGSLAPRQL¹⁵⁹ (Ac-Cys-FMDV peptide) is added. The reaction is stirred for 9 hours at room temperature and 70 mg of PEGA-NH₂ resin previously derivatized with N-succinimidyl 3-maleimidopropionate are added to remove the excess of peptide after 5 hours. The resin is removed by filtration and the solvent is lyophilized to provide carbon nanotube conjugate with two different peptides, one still protected.

The partially fully-protected peptide-carbon nanotube conjugate is treated with 1 ml of a 4 M HCl solution in dioxane. After stirring 1 hour, the product is obtained by precipitation in cold diethyl ether. Carbon nanotubes functionalized with the two different peptides (D′) are characterized by TEM microscopy and NMR spectroscopy.

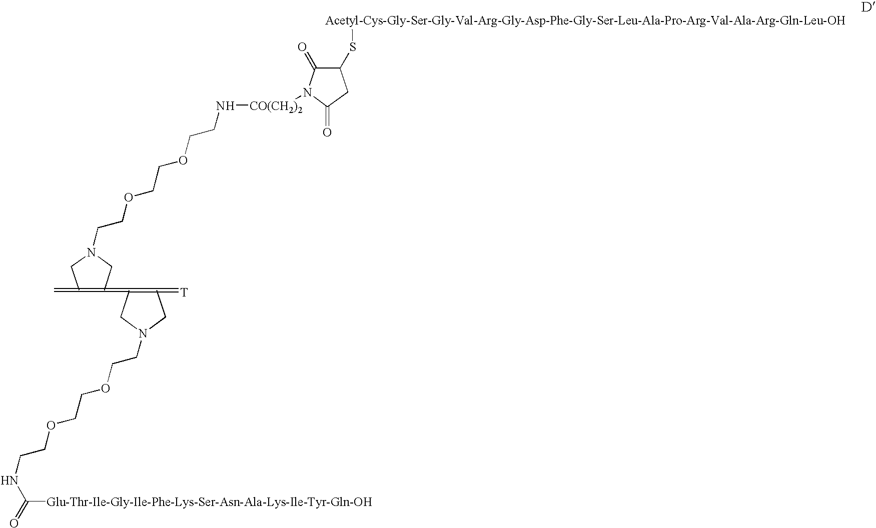

### Example 12

### Ex Vivo Assessment of the Capacity of Functionalized Carbon Nanotubes to Penetrate into Human Cells

Cytofluorometry

Murine 3T3 cells (ATCC CCL-92) were plated in 6 wells plate using RPMI 1640 STABILIX (Biomedia®, Boussens, France) modified medium (10% calf foetal serum (CFS), 1% non-essential amino acids, 0.05% β-mercaptoethanol, 0.1% gentamycin and 1% HEPES). After one night of incubation at 37° C. with 5% CO₂, the cells were incubated with a solution of FITC functionalized nanotube of formula (L) (1 μM, 5 μM and 10 μM, respectively) for 1 hour. The cells were washed, detached using a tnypsin solution (Biomedia®, Boussens, France) and collected by centrifugation at 1100 rpm. The cells were washed three times with an annexin V buffer solution (Pharmingen, Le Pont de Claix, France). 100 μL of the same buffer and 0.5 μL of annexin V APC (allophycocyanin) were added to the cells and incubated for 15 min. in the dark. Then, 5 μL of propidium iodide staining solution (50 μg/ml) was added. The analysis was performed using a cytofluorimetry machine FACSCalibur (Becton-Dickinson, Le Pont de Claix, France) operating on two different excitation wavelengths (543 nm and 647 nm). CellQuest® software (Becton-Dickinson, Le Pont de Claix, France) is used for the data analysis. The data obtained indicate that the FITC functionalized nanotube readily penetrate into 3T3 cells.

Fluorescent and Confocal Microscopy

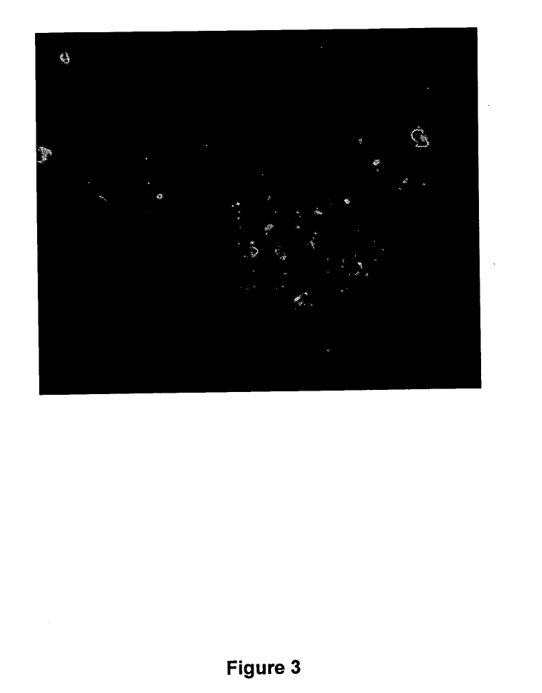

Murine 3T3 cells (ATCC CCL-92) were plated in RPMI 1640 STABILIX modified medium (10% CFS, 1% non-essential amino acids, 0.05% β-mercaptoethanol, 0.1% gentamycin and 1% HEPES). Glasses coverslips were covered with 2.5×10⁴ cells. After 2 hours, the cell culture medium was discarded and the coverslips washed with phosphate buffered saline (PBS). FITC functionalized carbon nanotubes (L) were overlayed on the cells at different concentration (1 μM, 5 μM and 10 μM respectively) and incubated for 5, 10 or 15 min. Then, 0.5 ml of cell culture medium was added and incubated for the time required depending on the experiment, at 37° C. with 5% CO₂. At the end of the incubation time, the cell medium was discarded and the cells washed once with PBS. The cells were fixed with 3.7% formalin for 10 min. and washed with PBS. The coverslip was dried and deposited on a microscope slide (76×26 mm) using 3 drops of commercial antifade agent (Dako, Carpinteria, USA). The coverslips were analysed on an Axioskop II fluorescent microscope (Zeiss, Le Pecq, France) using objectives 63× immersed in oil and 40× in the air. The coverslip were also analyzed on an Axiovert 100M confocal microscope (Zeiss, Le Pecq, France). The fluorescent microscopy picture of FIG. 3 shows that FITC functionalized carbon nanotubes have penetrated into 3T3 cells.

### Example 13

### In Vitro Assessment of the Immunological Reactivity of Peptide Functionalized Carbon Nanotubes

The immunological reactivity of the peptide functionalized carbon nanotube of structure (J), with the specific monoclonal antibody (mAb) 21×27, was assessed using surface plasmon resonance technology (Baird C. L. et al., J. Mol. Recognit. (2001) 14:261-268) on a Biacore 3000 instrument (Biacore, Uppsala, Sweden) (mAb 21×27 has been generated after injecting mice with the foot-and-mouth disease virus VP1 protein 147-156 peptide; this shorter peptide sequence is comprised in the 141-159 FMDV peptide and is able to induce antibodies which are cross reactive with the 141-159 peptide upon immunization in rice). This device measures the increase in mass on a coated gold film when interaction occurs between an immobilized ligand and an analyte in constant-flow over the surface. Prior to injecting the solution of free peptide (FMDV) or of peptide functionalized carbon nanotube (J), the specific mAb was immobilized on a chip. Briefly, rabbit anti-mouse Fcγ IgG (Biacore, Uppsala, Sweden) was immobilized on a CM5 carboxylated dextran coated chip by the standard amino-coupling procedure recommended by Biacore. Supernatants of hybridoma cultures secreting the anti-peptide mAb and a control monoclonal antibody of the same isotype were allowed to adsorb for 5 min at a flow rate of 5 μL/min to prepare the experimental channel and the control channel on the chip, respectively. The adsorption step was followed by the injection of the analytes (solvent, control carbon nanotube of formula (K), peptide-carbon nanotube of formula (J) and peptide (FMDV) in HBS (NaCl 150 mM, Hepes 10 mM pH=7.4, NP20 at 0.005%)) at a flow rate of 30 μL/min for 4 min followed by a dissociation phase of 5 min. The anti-mouse Fcγ ligand was regenerated by a 10 mM HCl solution passing for 30 seconds over the two channels. The results were corrected by subtracting from the experimental sensorgram that obtained with the control antibody to take into account non-specific interactions and by subtracting the experimental sensorgram obtained with the solvent to take into account the differential dissociation rate of the two monoclonal antibodies from the anti-mouse Fcγ IgG.

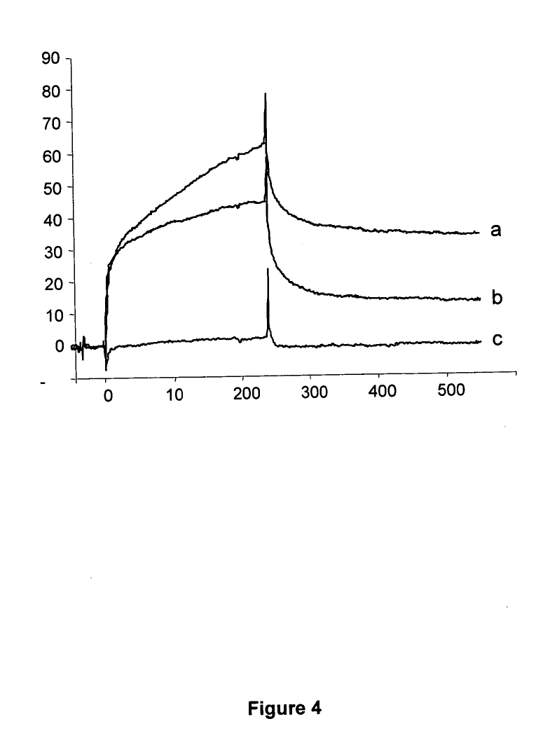

As shown in FIG. 4, the antibody recognized the FMDV peptide covalently linked to the carbon nanotube in a similar way as the free peptide. The slower association rate and the higher response in resonance units were due to the increase in molecular weight of the peptide-carbon nanotube complex compared to the free peptide. This was because the increase in response was directly correlated to the mass of the recognized molecule.

In addition, an Enzyme-Linked Immunosorbent Assay (ELISA) was performed to compare the recognition of carbon nanotube-conjugated or free FMDV peptide directly coated onto plastic wells by a polyclonal mouse anti-FMDV peptide serum (the polyclonal serum has been generated after injecting mice with the foot-and-mouth disease virus VP1 protein 141-159 peptide as described in Rowlands D. J. et al. Nature (1983) 306:694-697) or the mAb 21×27. Briefly, polyvinyl (Falcon, Franklin Lake, USA) or Maxisorp microtiter plates (Becton-Dickinson, Le Pont de Claix, France) were coated with the FMDV peptide, the peptide functionalized carbon nanotube of formula (J) and the reactive functionalized carbon nanotube of formula (C), as control, in carbonate/bicarbonate buffer at pH=9.6 overnight at 4° C. After washings with PBS containing 0.05% Tween (v/v) (PBS-T), plates were blocked with 1% bovine serum albumin (BSA) in PBS-T for 2 hours at 37° C. Serial two-fold dilutions of serum samples in PBS-T containing 0.3% BSA were made across the plate and the plates were incubated for 1 hour at 37° C. After washing, 50 μL of horseradish peroxidase-conjugated goat anti-mouse IgG ( 1/20000 in PBS-T) Fc-specific (Nordic Immunological Laboratories, Tilburg, The Netherlands) were added in each well and plates were incubated at 37° C. for 1 hour. Unbound conjugate was removed by washing with PBS-T. Finally peroxidase activity was evaluated by incubation with a buffer containing 3,3′,5,5′-tetramethylbenzidine (TMB, 150 μl per well). Two solutions were mixed before incubation: i) 80% (v/v) of a citrate buffer containing Na₂HPO₄H₂O 70 mM4, citric acid 30 mM, at pH 5 and ii) 20% (v/v) of a TMB solution containing 0.3% TMB (w/v), 72% DMSO (v/v), 18% glycerol (v/v), 10% citrate buffer. A catalytic amount of H₂O₂ was added followed by 15 min. incubation. Reaction was blocked by adding 50 μl of HCl 1M per well. The absorbance was measured at 450 nm.

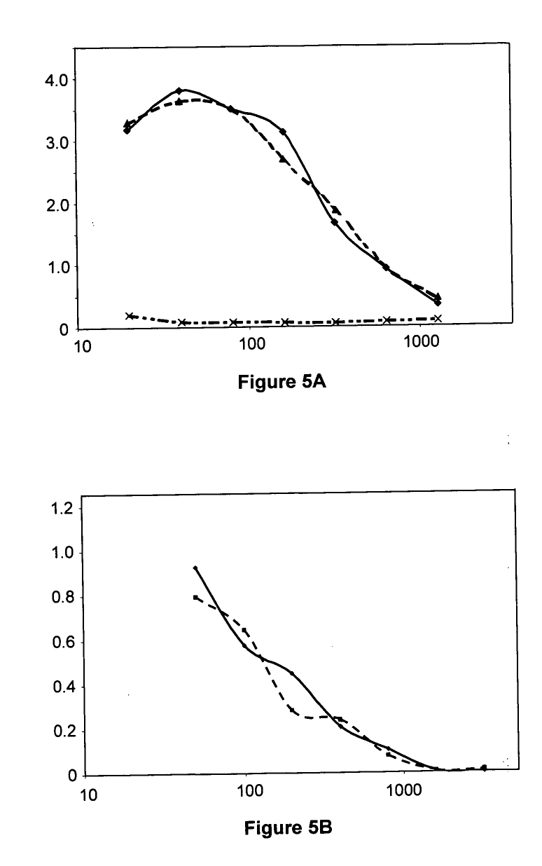

Both the free FMDV peptide and the peptide functionalized carbon nanotube (J) were recognized equally well by the polyclonal and monoclonal antibodies (see FIG. 5A and FIG. 5B). This is in agreement with the Biacore results and strongly suggests that the secondary structure of the nanotube-linked peptide, necessary for the spatial interaction with specific antibodies, is properly presented by the functionalized carbon nanotubes.

### Example 14

### In Vivo Assessment of the Immunological Reactivity of Peptide Functionalized Carbon Nanotubes

BALB/c mice (6-8 weeks old) were co-immunized intra-peritoneally (i.p.) with 100 μg, of FMDV 141-159 peptide either free (N-terminal acetylated) or attached to carbon nanotubes (formula J) together with 100 μg of ovalbumin (OVA) in a 1:1 emulsion in complete Freund's adjuvant. A booster injection was given i.p. in incomplete Freund's adjuvant three weeks later. Mice were bled at various time intervals after the boost and serum samples collected two weeks after the booster injection were tested for their anti-peptide antibody content. OVA was used to render the FMDV 141-159 peptide immunogenic, since it is not immunogenic when injected alone with an adjuvant in BALB/c mice (Francis M. J. Sci. Progress Oxford (1990) 74:115-130). Anti-peptide antibody responses were measured by ELISA according to the method described in Example 9, except that BSA-conjugated FMDV 141-159 peptide was used as solid-phase antigen (preliminary experiments have established that the use of BSA conjugated peptide as solid-phase antigen increased the sensitivity of the ELISA test as compared to the use of non-conjugated peptide), as well as the functionalized carbon nanotube of formula (H) as a control.

The results indicate that anti-FMDV 141-159 antibody response was enhanced when the peptide functionalized carbon nanotube of formula (J) was injected to mice in association with OVA as compared to when the free peptide was injected in association with OVA (FIG. 6). Moreover the observed antibody responses were peptide-specific and, as thus, were directed neither towards the functional group linking the FMDV 141-159 peptide to the carbon nanotube nor towards the carbon nanotubes in themselves (FIG. 6), thus showing that carbon nanotubes do not possess any intrinsic immunogenicity.

### Example 15

### Preparation of Cell Sections for TEM Analysis

SWNTs and MWNTs, from Carbon Nanotechnology, Inc. and Nanostructured & Amorphous Materials, Inc., respectively, were functionalized as described in Example 1.

HeLa cells (1.25×10⁵) were cultured into a 16-wells plate using DIEM medium at 37° C. and 5% CO₂ until 75% confluency. The cells were then incubated with a solution of 2.5 mg/ml of amino functionalized single- and/or multi-walled carbon nanotubes in PBS for 1 hour, washed twice with PBS and fixed for 2 hours at room temperature in 2.5% glutaraldehyde in a cacodilate buffer (sodium cacodilate 0.075 M, MgCl₂ 1 mM, CaCl₂ 1 mM, 4.5% sucrose pH 7.3). An amount of 10% v/v of a 1/10 saturated solution of picric acid in cacodilate buffer was then added into each wells and incubated overnight at 4° C. The specimen was washed three times for 15 minutes using distilled water, then treated with a 1% OsO₄ solution of in cacodilate buffer for 2 hours at room temperature. Cells were carefully rinsed with distilled water and post-fixed with a 2% solution of uranyl acetate in water overnight at 4° C. After several washes, the cells were dried with 70% and 90% ethanol for 10 minutes each, and twice with absolute ethanol for 20 minutes. An amount of fresh Epon® 812 resin was prepared as suggested by EMS (Electron Microscopy Sciences) technical data sheet and distributed into each well. The plate was stored into an oven at 65° C. for three days. Each resin block was then removed from the plastic support and cut. A Reichert-Jung Ultracut-E ultramicrotome with a diamond knife Ultramicrotomy® 45° was used to cut the cells embedded into the resin. A thickness value of 90 nm was chosen. Three subsequent slices were deposited onto a formvar grid and observed under an electronic transmission microscope Hitachi 600 at 75kV. Images were taken by an AMT high sensitive camera at different level of magnification.

The results shown in FIG. 7 for MWNTs indicate that the functionalized carbon nanotubes according to the invention are localized inside a cell.

### Example 16

### Flow Cytometry Measurements of Cell Viability

To analyse the cell viability by FACS, exponentially growing HeLa cells in DMEM supplemented with 10% (v/v) fetal calf serum, were incubated with a solution of amino functionalized single- (SWNT-NH₂) and/or multi-walled (MWNT-NH₂) carbon nanotubes at different concentration (0.01-1 mg/ml) for 6, 12 and 24 hours, respectively, rinsed and harvested with trypsin for 5 min at 37° C., centrifuged at 1000 tr/min for 10 min and washed three times with annexin V buffer solution. 100 μL of the same buffer and 0.5 μL of annexin V-FITC were added to the cells and incubated for 15 min in the dark. Then, 5 μL of propidium iodide staining solution (50 μg/ml) and 200 μL of annexin V buffer were added. The analysis was performed using a cytofluorimetry FACSCalibur® instrument operating at 494 nm and 647 nm excitation wavelength. CellQuest® software was used for the data analysis. A minimum of 40,000 events per sample were analysed.

The results shown in FIG. 8 indicate that functionalized carbon nanotubes according to the present invention are essentially non-toxic at concentrations corresponding to an in vivo use.

1. A functionalized carbon nanotube, the surface of which carries covalently bound reactive and/or activable functional groups which are homogeneously distributed on said surface, said functionalized carbon nanotube being substantially intact and soluble in organic and/or aqueous solvents.

2. A functionalized carbon nanotube according to claim 1, wherein said carbon nanotube is a single-walled (SWNT) or a multi-walled carbon nanotube (MWNT).

3. A functionalized carbon nanotube according to claim 2, wherein the organic solvents are selected from a group comprising dimethylformamide, dichloromethane, chloroform, acetonitrile, dimethylsulfoxide, methanol, ethanol, toluene, isopropanol, 1,2-dichloroethane, N-methylpyrrolidone, tetrahydrofuran.

4. A functionalized carbon nanotube according to claim 3, of following general formula:
[Cₙ]—Xₘ
wherein:
Cₙ are surface carbons of a substantially cylindrical carbon nanotube of substantially constant diameter, said diameter being from about 0.5 to about 50 nm, in particular from about 0.5 to 5 nm for SWNTs and from about 20 to about 50 nm for MWNTs,
X represents one or several functional groups, identical or different, each functional group comprising at least one effective group,
n is an integer from about 3.10³ to about 3.10⁶,
m is an integer from about 0.001 n to about 0.1 n,
there are from about 2.10⁻¹¹ moles to about 2.10⁻⁹ moles of X functional groups
per cm² of carbon nanotube surface.

5. A functionalized carbon nanotube according to claim 4, wherein X represents two different functional groups, X¹ and X², said functionalized nanotube corresponding to the following formula:
[Cₙ]—[X¹ₘ₁][X²ₘ₂]
wherein, independently from each other, m₁ and m₂ represent integer from about 0.001 n to about 0.1 n.

6. A functionalized nanotube according to claim 4, wherein X represents at least one functional group comprising two effective groups, identical or different.

7. A functionalized carbon nanotube according to claim 4, wherein X represents one or several substituted pyrrolidine rings, identical or different, of the following general formula (I): 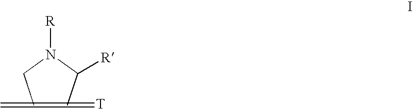
wherein T represents a carbon nanotube, and independently from each other R and R′ represent —H or a group of formula -M-Y-(Z)ₐ-(P)_{b}, wherein a represents 0 or 1 and b represents an integer from 0 to 8, preferably 0, 1, or 2, P representing identical or different groups when b is greater than 1, provided R and R′ cannot simultaneously represent H, and:
M is a spacer group from about 1 to about 100 atoms, such as a group selected from the list comprising —(CH₂)ᵣ— or —(CH₂—CH₂—O)ᵣ—CH₂—CH₂—, wherein r is an integer from 1 to 20;
Y is a reactive group when a=b=0, such as a group selected from the list comprising —OH, —NH₂, —COOH, —SH, —CHO, a ketone such as —COCH₃, an azide or a halide; or derived from a reactive group, when a or b is different from 0, such as a group selected from the list comprising —O—, —NH—, —COO—, —S—, —CH═, —CH₂—, —CCₖH₂ₖ₊₁═, wherein k is an integer from 1 to 10, in particular —CCH₃═, or —CHCₖH₂ₖ₊₁—, wherein k is an integer from 1 to 10, in particular —CHCH₃—;
Z is a linker group, liable to be linked to at least one P group, and if need be to release said P group, such as a group of one of the following formulae when a=1 and b=0: 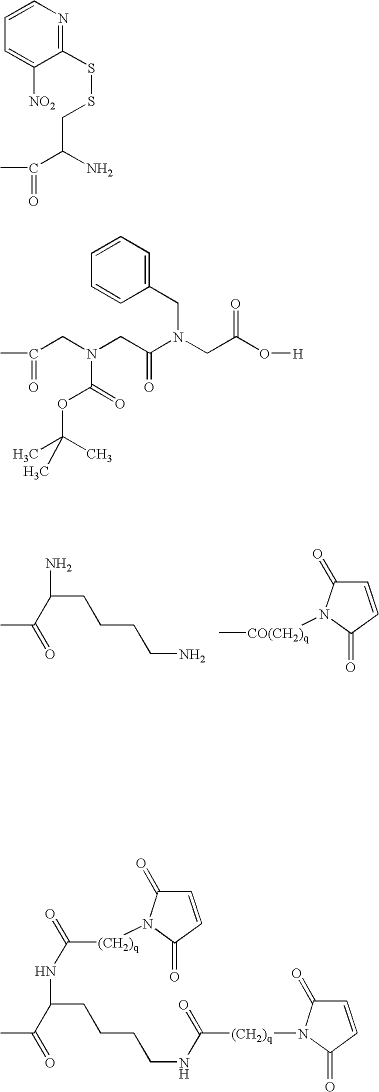
wherein q is an integer from 1 to 10;
or of one of the corresponding following formulae when a=1 and b=1 or 2: 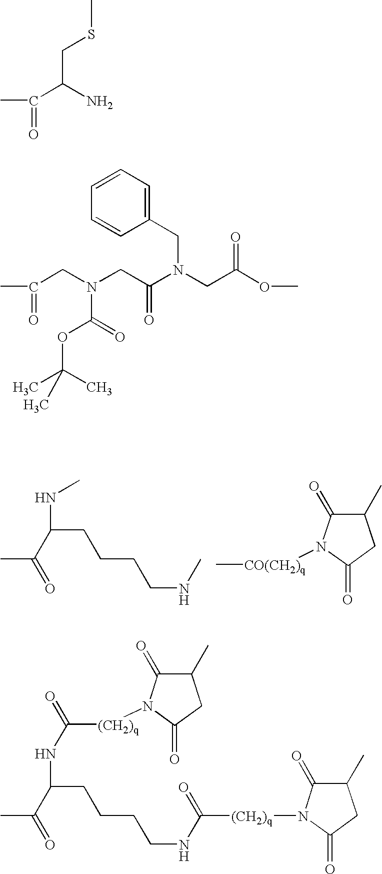
wherein q is an integer from 1 to 10;
P is an effective group allowing spectroscopic detection of said functionalized carbon nanotube, such as a fluorophore, such as FITC, a chelating agent, such as DTPA, or an active molecule, liable to induce a biological effect, such as an amino acid, a peptide, a pseudopeptide, a protein, such as an enzyme or an antibody, a nucleic acid, a carbohydrate, or a drug.
if appropriate at least one of Y, Z, or P groups, can be substituted by a capping group, such as CH₃CO— (acetyl), methyl, ethyl, or benzylcarbonyl, or a protecting group such as methyl, ethyl, benzyl, tert-butyl, trityl, 3-nitro-2-pyridylsulfenyl, tert-butyloxycarbonyl (Boc), fluorenylmethyloxycarbonyl (Fmoc), benzyloxycarbonyl (Cbz), benzoyl (Bz), trimethylsilylethyloxycarbonyl, phtalimide, dimethylacetal, diethylacetal or, 1,3-dioxolane.

8. A functionalized carbon nanotube according to claim 7, wherein X represents two different substituted pyrrolidine rings, of the following general formula (I′): 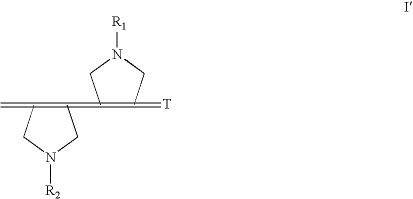
wherein T represents a carbon nanotube, R₁ and R₂ are different and represent, independently from each other, —H or a group of formula -M-Y-(Z)ₐ-(P)_{b}, M, Y, Z, P, a and b.

9. A functionalized carbon nanotube according to claim 7, wherein a=b=0 and Y is a reactive group selected from the list comprising —OH, —NH₂, —COOH, —SH, —CHO, a ketone, such as —COCH₃, an azide, or a halide, in particular —NH₂, said functionalized carbon nanotube being, if appropriate, substituted by a capping or a protecting group, in particular a Bz, Boc or acetyl group, and being for instance a functionalized carbon nanotube of one of the following formulae:

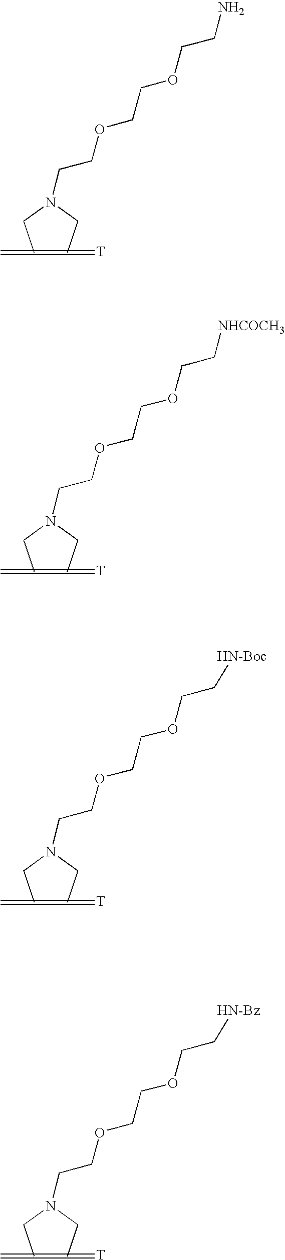

10. A functionalized carbon nanotube according to claim 7, wherein a=1 and b=0, Y is derived from a reactive group and selected from the list comprising —O—, —NH—, —COO—, —S—, —CH═, —CH₂—, —CCₖH₂ₖ₊₁═, wherein k is an integer from 1 to 10, in particular —CCH₃═, or —CHCₖH₂ₖ₊₁—, wherein k is an integer from 1 to 10, in particular —CHCH₃—, and Z represents in particular the group of the following formula: 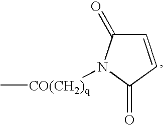
or of the following formula: 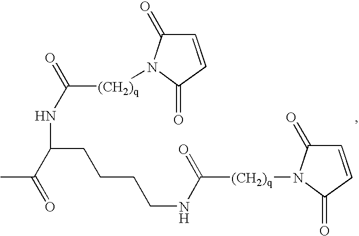
wherein q is an integer from 1 to 10, said functionalized carbon nanotube being if appropriate substituted by a protecting group, and being for instance the functionalized carbon nanotube of the following formula: 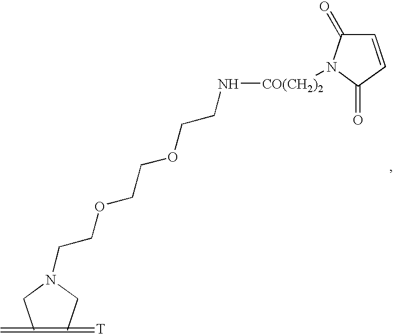
or of the following formula:

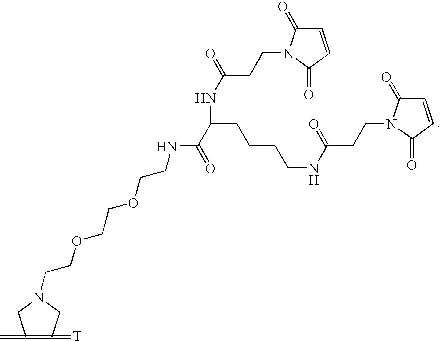

11. A functionalized carbon nanotube according to claim 7, wherein a=0 and b=1, Y is derived from a reactive group and selected from the list comprising —O—, —NH—, —COO—, —S—, —CH═, —CH₂—, —CCₖH₂ₖ₊₁=, wherein k is an integer from 1 to 10, in particular —CCH₃═, or —CHCₖH₂ₖ₊₁—, wherein k is an integer from 1 to 10, in particular —CHCH₃—, and P is an effective group or an active molecule, in particular FITC, an amino acid, such as glycine, a peptide, such as the peptide H-Lys-Gly-Tyr-Tyr-Gly-OH, or DTPA, said functionalized carbon nanotube being if appropriate substituted by a protecting group, such as Fmoc, and being for instance a functionalized carbon nanotube of one of the following formulae:

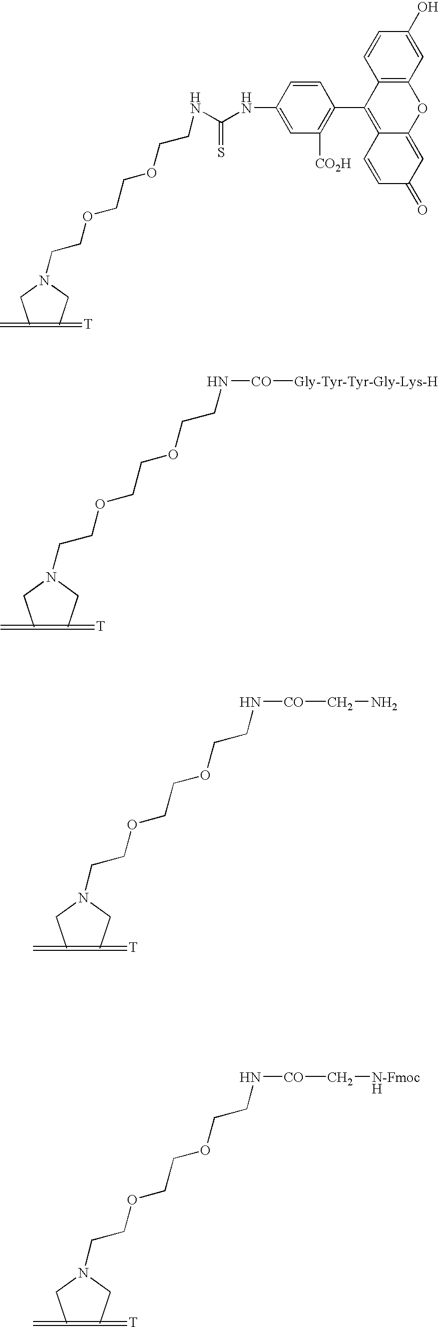

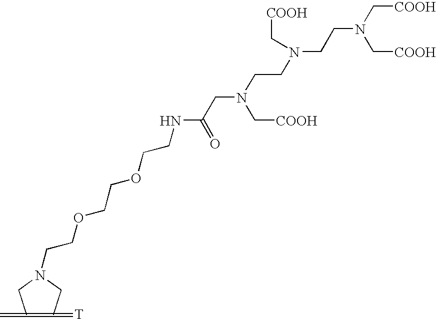

12. A functionalized carbon nanotube according to claim 7, wherein a=1 and b=1, Y is derived from a reactive group and selected from the list comprising —O—, —NH—, —COO—, —S—, —CH═, —CH₂—, —CCₖH₂ₖ₊₁=, wherein k is an integer from 1 to 10, in particular —CCH₃═, or —CHCₖH₂ₖ₊₁—, wherein k is an integer from 1 to 10, in particular —CHCH₃—, Z represents in particular the group of the following formula: 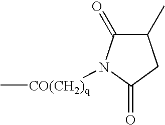
wherein q is an integer from 1 to 10, and P is in particular a peptide, such as the peptide Acetyl-Cys-Gly-Ser-Gly-Val-Arg-Gly-Asp-Phe-Gly-Ser-Leu-Ala-Pro-Arg-Val-Ala-Arg-Gln-Leu-OH, said functionalized carbon nanotube being if appropriate substituted by a protecting group, and being for instance the functionalized carbon nanotube of the following formula:

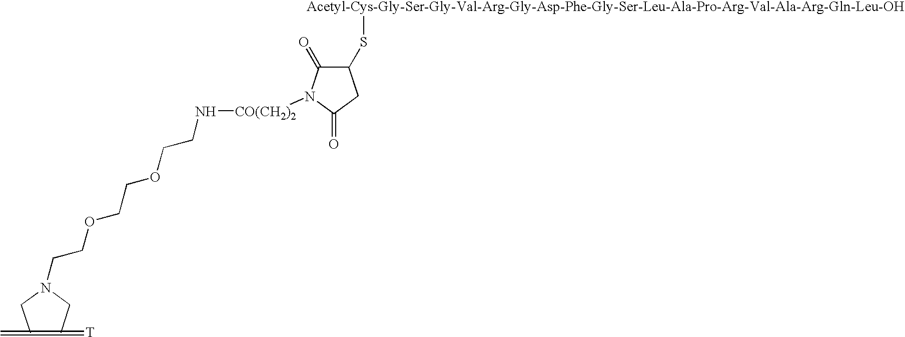

13. A functionalized carbon nanotube according to claim 7, wherein a=1 and b=2, that is wherein independently of each other R and R′ represent —H or a group of formula M-Y-Z-(P)₂ or M-Y-Z-(PP′), provided R and R′ cannot simultaneously represent —H, Y is derived from a reactive group and selected from the list comprising —O—, —NH—, —COO—, —S—, —CH═, —CH₂—, —CCₖH₂ₖ₊₁═, wherein k is an integer from 1 to 10, in particular —CCH₃═, or —CHCₖH₂ₖ₊₁—, wherein k is an integer from 1 to 10, in particular —CHCH₃—, Z represents in particular the group of the following formula: 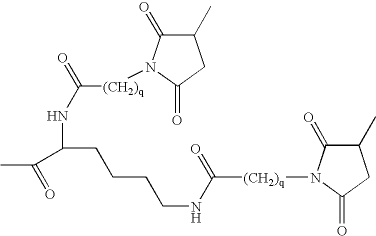
wherein q is an integer from 1 to 10, P and P′ are different and represent an effective group, in particular P and P′ represent a peptide, such as the peptide Acetyl-Cys-Gly-Ser-Gly-Val-Arg-Gly-Asp-Phe-Gly-Ser-Leu-Ala-Pro-Arg-Val-Ala-Arg-Gln-Leu-OH, said functionalized carbon nanotube being if appropriate substituted by a protecting group, and being for instance the functionalized carbon nanotube of the following formula:

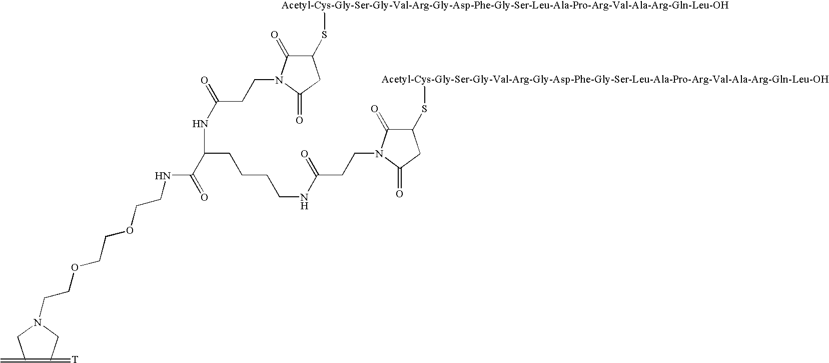

14. A functionalized carbon nanotube according to claim 11, wherein P is a peptide or a protein, said peptide or protein comprising in particular a B cell epitope or a T cell epitope, such as a T helper epitope or a T cytotoxic epitope, or a mixture thereof.

15. A process for preparing a functionalized carbon nanotube of the following formula I: 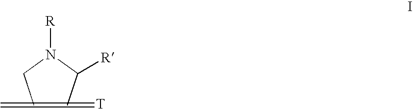
wherein T represents a carbon nanotube and independently from each other R and R′ represent —H or a group of formula -M-Y, provided R and R′ cannot simultaneously represent H, wherein:
-M- is a spacer group from about 1 to about 100 atoms, such as a group selected from the list comprising —(CH₂)ᵣ— or —(CH₂—CH₂—O), —CH₂—CH₂—, wherein r is an integer from 1 to 20;
—Y is a reactive group, such as a group selected from the list comprising, —OH, —NH₂, —COOH, —SH, —CHO, a ketone such as —COCH₃, an azide, a halide, if appropriate protected, such as —O-Q, —NH-Q, —COO-Q, —S-Q, —CH(OQ)₂, 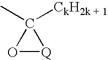
wherein k is an integer from 1 to 10, in particular 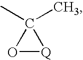
wherein Q is a protecting group or forms a protecting group with the adjacent atoms to which it is linked;
said process comprising the following step:
adding, to a carbon nanotube, the compounds R′—CHO and R—NH—CHR″—COOR′″ by a 1,3-dipolar cycloaddition, wherein:
R and R′ are as defined above;
R″ is —H or an amino acid side-chain;
R′″ is —H, an alkyl group of 1 to 5 carbon atoms, a (CH₂CH₂O)ₜ—CH₃
group, wherein t is an integer from 1 to 20, or an aromatic group;
to obtain a functionalized carbon nanotube of formula I, if appropriate protected;
if necessary, deprotecting the functionalized carbon nanotube of formula I, to obtain an unprotected functionalized carbon nanotube of formula I.

16. A process for preparing a functionalized carbon nanotube of the following formula I′: 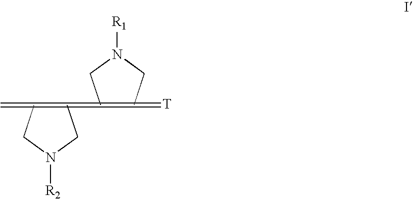
wherein T represents a carbon nanotube, R₁ and R₂ are different and represent independently from each other —H or a group of formula -M-Y, M, and Y being as defined in claim 15,
said process comprising the following step:
adding to a carbon nanotube by a 1,3-dipolar cycloaddition, a mixture of the compounds (CH₂O)ₙ (paraformaldehyde), R₁—NH—CH₂—COOH and R₂—NH—CH₂—COOH, wherein R₁ and R₂ are different and represent independently from each other —H or a group of formula -M-Y, to obtain a functionalized carbon nanotube of formula I′, if appropriate protected, R₁ and R₂ carrying similar or different protecting groups,
if necessary, deprotecting R₁ and/or R₂.

17. A process for preparing a functionalized carbon nanotube of the following formula I: 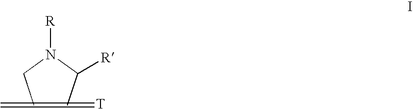
wherein T represents a carbon nanotube and independently from each other R and R′ represent —H or a group of formula -M-Y-Z, provided R and R′ cannot simultaneously represent —H, wherein:
-M- is a spacer group from about 1 to about 100 atoms, such as a group selected from the list comprising —(CH₂)ᵣ— or —(CH₂—CH₂—O)ᵣ—CH₂—CH₂—, wherein r is an integer from 1 to 20;
—Y— is a group derived from a reactive group, such as a group selected from the list comprising, —O—, —NH—, —COO—, —S—, —CH═, —CH₂—, —CCₖH₂ₖ₊₁═, wherein k is an integer from 1 to 10, in particular —CCH₃═, or —CHCₖH₂ₖ₊₁—, wherein k is an integer from 1 to 10, in particular —CHCH₃—;
-Z is a linker group, liable to be linked to at least one P group, and if need be to release said P group, if appropriate protected by one or several capping or protecting groups -Q, identical or different, such as a group of one of the following formulae: 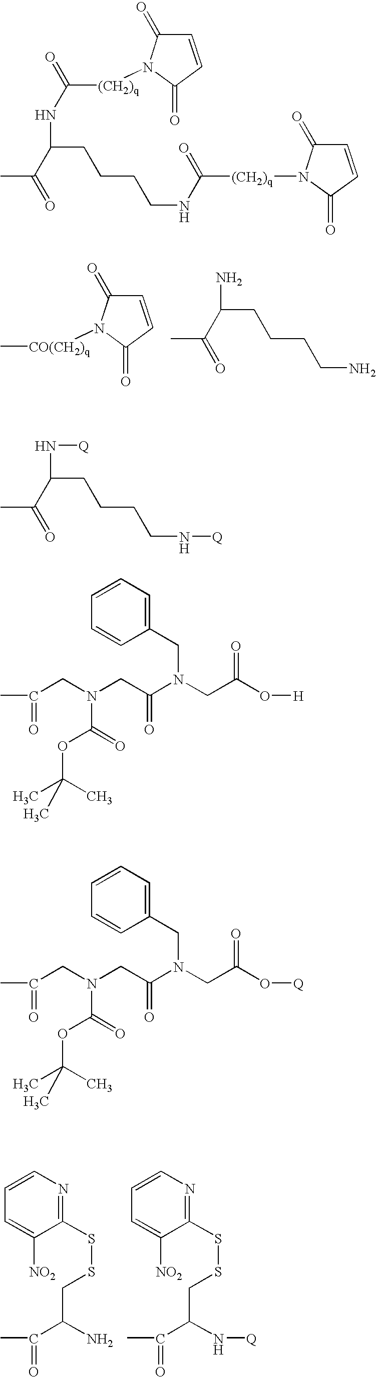
wherein q is an integer from 1 to 10;
said process comprising the following steps:
adding to a unprotected functionalized carbon nanotube of formula I according to claim 15 a linker group of formula Z, if appropriate protected by one or several capping or protecting group -Q, identical or different, such as a group of one of the following formulae: 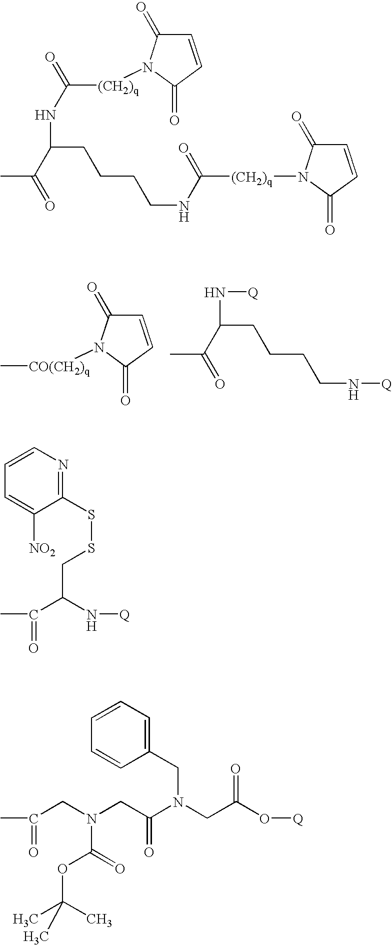
wherein q is an integer from 1 to 10;
to obtain a functionalized carbon nanotube of formula I, if appropriate protected;
if necessary, deprotecting the functionalized carbon nanotube of formula I, to obtain an unprotected functionalized carbon nanotube of formula I.

18. A process for preparing a functionalized nanotube of the following formula I: 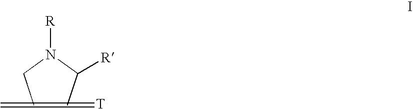
wherein T represents a carbon nanotube and independently from each other R and R′ represent —H or a group of formula -M-Y-Z-P or of formula -M-Y—P, provided R and R′ cannot simultaneously represent —H, wherein:
-M- is a spacer group from about 1 to about 100 atoms, such as a group selected from the list comprising —(CH₂)ᵣ— or —(CH₂—CH₂—O)ᵣ—CH₂—CH₂—, wherein r is an integer from 1 to 20;
—Y— is a group derived from a reactive group, such as a group selected from the list comprising, —O—, —NH—, —COO—, —S—, —CH═, —CH₂—, —CCₖH₂ₖ₊₁═, wherein k is an integer from 1 to 10, in particular —CCH₃═, or —CHCₖH₂ₖ₊₁—, wherein k is an integer from 1 to 10, in particular —CHCH₃—;
-Z- is a linker group, liable to be linked to a P group, and if need be to release said P group, such as a group of one of the following formulae: 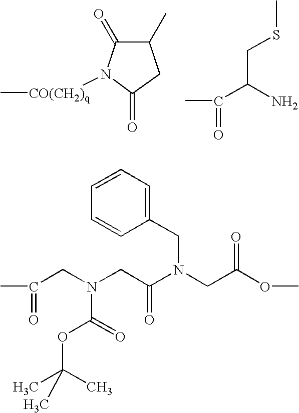
wherein q is an integer from 1 to 10;
—P is an effective group allowing spectroscopic detection of said functionalized carbon nanotube, such as a fluorophore, such as FITC, a chelating agent, such as DTPA, or an active molecule, liable to induce a biological effect, if appropriate protected, such as an amino acid, a peptide, a pseudopeptide, a protein, such as an enzyme or an antibody, a nucleic acid, a carbohydrate, or a drug;
said process comprising the following steps:
adding to an unprotected functionalized carbon nanotube of formula I according to claim 15, an effective group or an active molecule of formula P, if appropriate protected such as a fluorophore, such as FITC, a chelating agent, such as DTPA, an amino acid, a peptide, a pseudopeptide, a protein, such as an enzyme or an antibody, a nucleic acid, a carbohydrate, or a drug, or adding to an unprotected functionalized carbon nanotube of formula I, a group of formula Z-P, if appropriate protected,
to obtain a functionalized carbon nanotube of formula I, if appropriate protected;
if necessary, deprotecting the functionalized carbon nanotube of formula I, to obtain an unprotected functionalized carbon nanotube of formula I.

19. A process for preparing a peptide or protein functionalized carbon nanotube, of the following formula I: 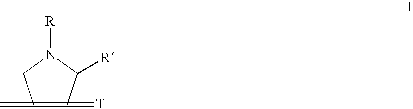
wherein T represents a carbon nanotube and independently from each other R and R′ represent H or a group of formula -M-Y—P, or of formula -M-Y-Z, provided R and R′ cannot simultaneously represent —H, wherein:
-M- is a spacer group from about 1 to about 100 atoms, such as a group selected from the list comprising —(CH₂)ᵣ— or —(CH₂—CH₂—O)ᵣ—CH₂—CH₂—, wherein r is an integer from 1 to 20;
—Y— is a group derived from a reactive group, such as a group selected from the list comprising, —O—, —NH—, —COO—, —S—, —CH═, —CH₂—, —CCₖH₂ₖ₊₁═, wherein n is an integer from 1 to 10, in particular —CCH₃═, or —CHCₖH₂ₖ₊₁—, wherein k is an integer from 1 to 10, in particular —CHCH₃—;
-Z- is a linker group, in particular a group of the following formula: 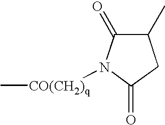
wherein q is an integer from 1 to 10;
—P is a peptide, in particular of following formula: —[OC—CHAᵢ-NH]ₜ—H, wherein -Aᵢ is an amino acid side-chain, i is an integer from 1 to t and t is an integer from 1 to 150, advantageously from 1 to 50;
said process comprising the following steps:
adding to a functionalized carbon nanotube of formula I, according to claim 15, a protected amino acid of the following formula:
Q-NH—CHAᵢ-COOH
wherein -Aᵢ is as defined above and -Q is a protecting group to obtain a functionalized carbon nanotube of the following formula II: 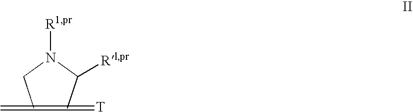
wherein independently from each other R^{l,pr} and R′^{l,pr} represent —H or a group of formula -M-Y—OC—CHAᵢ-NH-Q, or of formula -M-Y-Z-OC—CHAᵢ-NH-Q, wherein -M-, —Y—, -Z-, -Aᵢ and -Q are as defined above;
deprotecting the functionalized carbon nanotube of formula II to obtain a functionalized carbon nanotube of the following formula III: 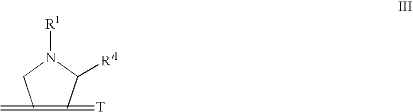
wherein independently from each other R^{l} and R′^{l} represent —H or a group of formula -M-Y—OC—CHA₁-NH₂, or of formula -M-Y-Z-OC—CHAᵢ-NH₂, wherein -M-, —Y—, -Z-, and -Aᵢ are as defined above;
adding to the functionalized carbon nanotube obtained at the preceding step a protected amino acid of the following formula:
Q-NH—CHAᵢ-COOH
wherein -Aᵢ is as defined above and -Q is a protecting group to obtain a functionalized carbon nanotube of the following formula IV: 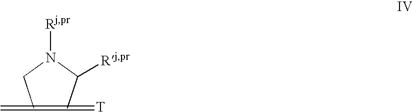
wherein independently from each other R^{j,pr} and R′^{j,pr} represent —H or a group of formula -M-Y—[OC—CHAᵢ-NH]ⱼ-Q, or of formula -M-Y-Z-[OC—CHAᵢ-NH]ⱼ-Q, wherein -M-, —Y—, -Z-, -Aᵢ and -Q are as defined above, and j is an integer from 2 to t;
deprotecting the functionalized carbon nanotube of formula IV to obtain a functionalized carbon nanotube of the following formula V: 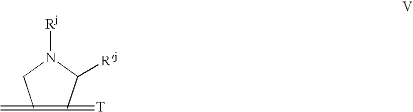
wherein independently from each other R^{j} and R′^{j} represent —H or a group of formula -M-Y—[OC—CHAᵢ-NH]ⱼ—H, or of formula M-Y-Z-[OC—CHAᵢ-NH]ⱼ—H, wherein -M-, —Y—, -Z-, and -Aᵢ are as defined above, and j is an integer from 2 to t;
repeating the last two steps t-1 times to obtain a peptide or protein functionalized carbon nanotube of formula I.

20. A process according to claim 15, wherein -Q is a capping group, such as CH₃CO— (acetyl), methyl, ethyl, or benzylcarbonyl, or a protecting group, such as a group selected from the list comprising methyl, ethyl, benzyl, tert-butyl, trityl, 3-nitro-2-pyridylsulfenyl, tert-butyloxycarbonyl (Boc), fluorenylmethyloxycarbonyl (Fmoc), benzyloxycarbonyl (Cbz), benzoyl (Bz), trimethylsilylethyloxycarbonyl, phtalimide, or ethyleneoxy.

21. A process for preparing a functionalized carbon nanotube of one of the following formulae VI, VII and VII′: 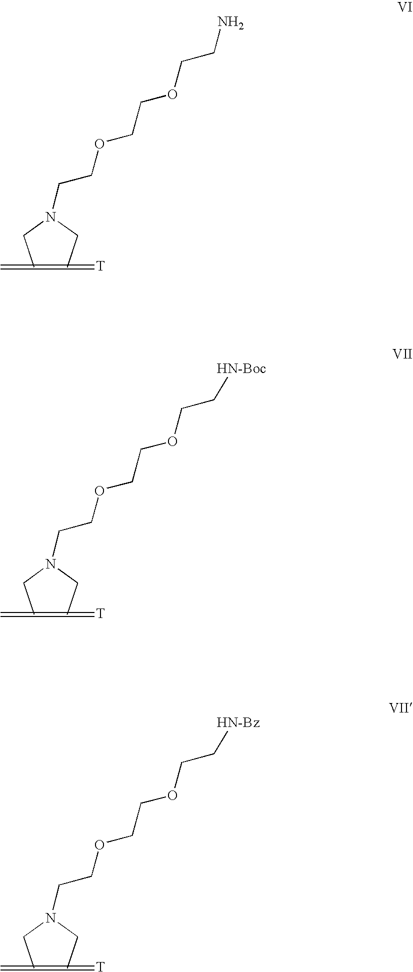
wherein T represents a carbon nanotube, Boc represents tert-butyloxycarbonyl and Bz represents benzoyl, said process comprising the following steps:
adding, to a carbon nanotube, the compounds (CH₂O)ₙ (paraformaldehyde) and Boc-NH—(CH₂—CH₂—O)₂—CH₂—CH₂—NH—CH₂—COOH or Bz-NH—(CH₂—CH₂—O)₂—CH₂—CH₂—NH—CH₂—COOH by a 1,3-dipolar cycloaddition, to obtain a protected functionalized carbon nanotube of respective formula VII or VII′;
if necessary, deprotecting the protected functionalized carbon nanotube of formula VII or VII′, to obtain an unprotected functionalized carbon nanotube of formula VI.

22. A process for preparing a functionalized carbon nanotube of the following formula VIII: 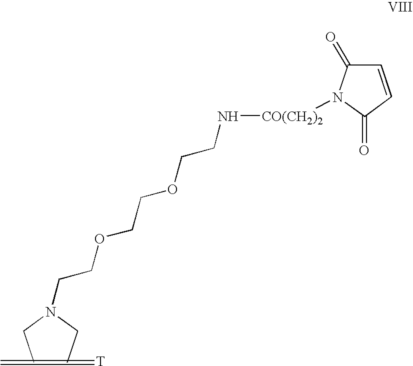
wherein T represents a carbon nanotube, said process comprising the following step:
adding, to a carbon nanotube of formula VI according to claim 21, a compound of the following formula: 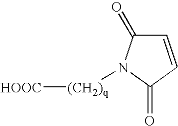
to obtain a functionalized carbon nanotube of formula VIII.

23. A process for preparing a functionalized carbon nanotube of one of the following formulae IXa, IXb, IXc, IXd, IXe, IXf, IXg, Xb, Xc and Xf: 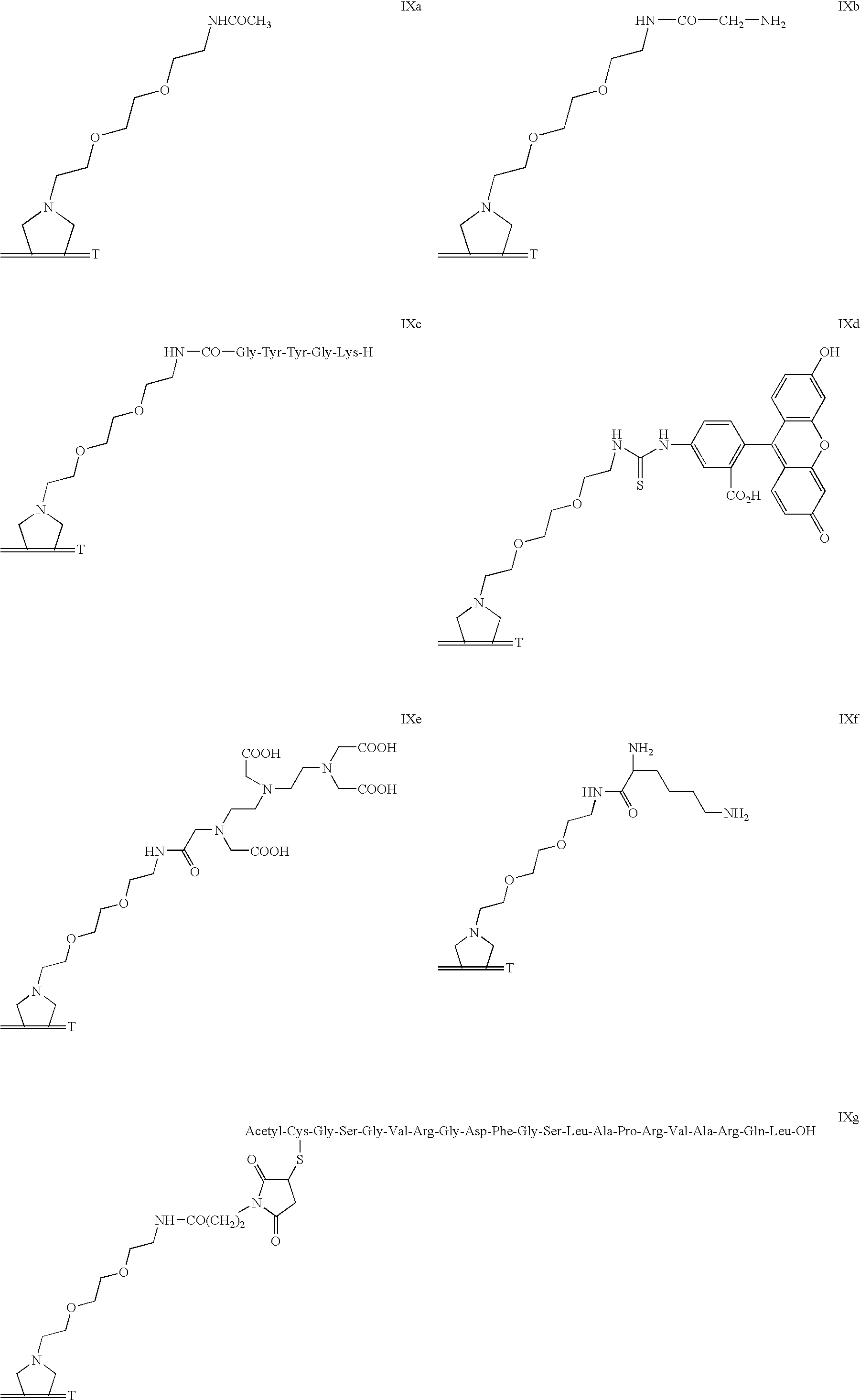 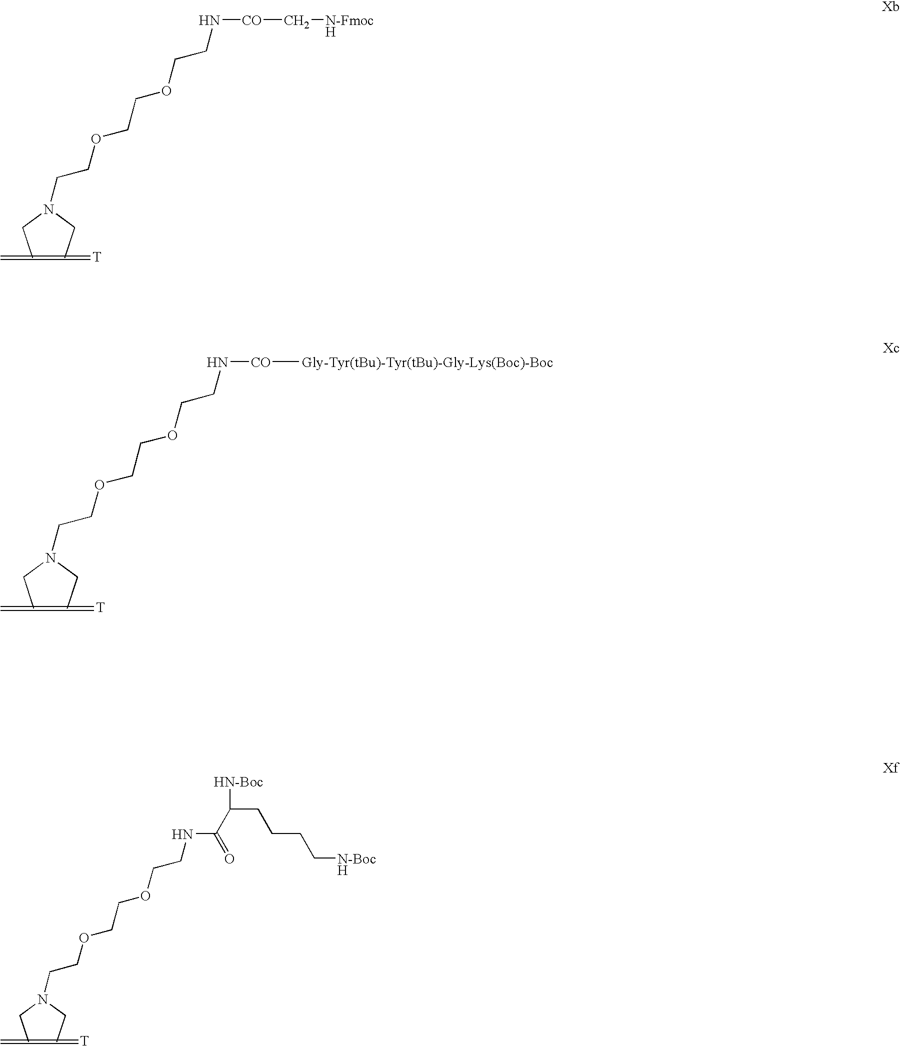
wherein T represents a carbon nanotube, Fmoc represents fluorenylmethyloxycarbonyl, tBu represents tert-butyl and Boc represents tert-butyloxycarbonyl, said process comprising the following steps:
adding,
either to a functionalized carbon nanotube of formula VI according to claim 21, a group chosen among: CH₃—COOH, Fmoc-Gly-OH, Boc-Lys(Boc)-Gly-Tyr(tBu)-Tyr(tBu)-Gly-OH, FITC, DTPA or Boc-Lys(Boc)-OH,
or to a functionalized carbon nanotube of formula VIII, the following group, Acetyl-Cys-Gly-Ser-Gly-Val-Arg-Gly-Asp-Phe-Gly-Ser-Leu-Ala-Pro-Arg-Val-Ala-Arg-Gln-Leu-OH,
to obtain a functionalized carbon nanotube of respective formula IXa, Xb, Xc, IXd, IXe, IXf or IXg;
if necessary, deprotecting the functionalized carbon nanotube of formula Xb, Xc, or Xf, to obtain respectively the functionalized carbon nanotube of formula IXb, IXc, or IXf.

24. A process for preparing a functionalized carbon nanotube of the following formulae XIa and XIb: 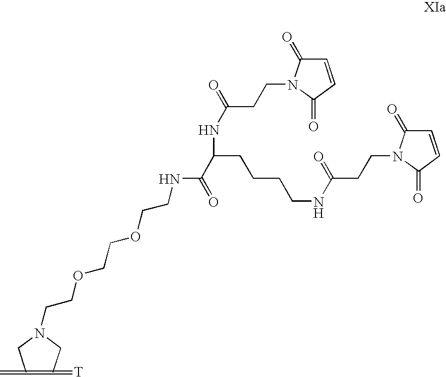 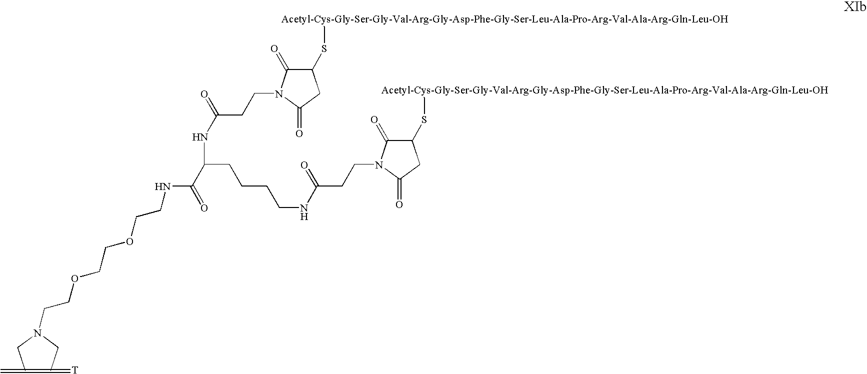
wherein T represents a carbon nanotube, said process comprising the following step:
adding, to a carbon nanotube of formula IXf according to claim 23, a compound of the following formula: 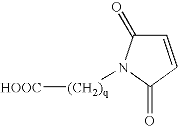
to obtain a functionalized carbon nanotube of formula XIa,
optionally adding to the functionalized nanotube of formula XIa the following group, Acetyl-Cys-Gly-Ser-Gly-Val-Arg-Gly-Asp-Phe-Gly-Ser-Leu-Ala-Pro-Arg-Val-Ala-Arg-Gln-Leu-OH, to obtain a functionalized carbon nanotube of formula XIb.

25. A functionalized carbon nanotube such as obtained by the process of claim 15.

26. A pharmaceutical composition comprising as active substance at least one functionalized carbon nanotube according to claim 1, said functionalized carbon nanotube being non-toxic, in association with a pharmaceutically acceptable vehicle, such as a liposome, a cyclodextrin, a microparticle, a nanoparticle, or a cell penetrating peptide.

27. A method of transport of pharmaceutically active molecules comprising the use of a functionalized carbon nanotube according to claim 1.

28. A method of delivery of drugs, in particular of intracellular delivery of drugs, comprising the use of an appropriate amount of a functionalized carbon nanotube according to claim 1.

29. A method of preparation of an immunogenic composition intended to provide an immunological protection to the individual to whom it has been administrated, comprising the use of an appropriate amount of a functionalized carbon nanotube according to claim 1.

30. A method for the treatment or the prophylaxis of cancer, autoimmune or infectious diseases, comprising the administration of an appropriate amount of a functionalized carbon nanotube according to claim 1.

31. A method of preparation of functionalized surfaces such as plastic or glass surfaces comprising the use of a functionalized carbon nanotube according to claim 1.

32. A method of preparation of electrochemical biosensors comprising the use of a functionalized carbon nanotube according to claim 1.
